(19) United States
(12) Patent Application Publication
(10) Pub. No.: US 2011/0108411 A1
(43) Pub. Date: May 12, 2011

(54) METHODS FOR LABELING A SUBSTRATE USING A HETERO-DIELS-ALDER REACTION

(76) Inventors: VLADIMIR V. POPIK, Watkinsville, GA (US); Selvanathan Arumugam, Athens, GA (US)

(21) Appl. No.: 12/945,328

(22) Filed: Nov. 12, 2010

Related U.S. Application Data
(60) Provisional application No. 61/260,102, filed on Nov. 11, 2009.

(51) Int. Cl. B01J 19/08; C07C 39/38; C07C 49/796

(52) U.S. Cl. 204/157.69; 568/737; 568/328

(57) ABSTRACT

Methods for labeling a substrate using a hetero-Diels-Alder reaction are disclosed. The hetero-Diels-Alder reaction includes the reaction of an o-quinone methide (e.g., an o-naphthoquinone methide) with a polarized olefin to form a hetero-Diels-Alder adduct. The o-quinone methide or the polarized olefin can be attached to a surface of a substrate, and the other of the o-quinone methide or the polarized olefin can include a detectable label. The o-quinone methide can conveniently be generated by irradiation of a precursor compound, preferably in an aqueous solution, suspension, or dispersion.

The present application claims the benefit of U.S. Provisional Application No. 61/260,102, filed Nov. 11, 2009, which is incorporated herein by reference in its entirety.

### GOVERNMENT FUNDING

The present invention was made with government support under Grant No. CHE 0842590, awarded by the National Science Foundation. The Government has certain rights in this invention.

### BACKGROUND

Connection (or ligation in biochemistry) of two or more substrates or immobilization of various compounds are often achieved with the help of “click chemistry,” which describes a set of bimolecular reactions that are modular, wide in scope, high yielding, create only inoffensive by-products, are stereospecific, simple to perform and that require benign or easily removed solvent. Although meeting all of the above requirements is difficult to achieve, several processes have been identified as corning very close to the ideal “click reaction.” Among them are 1,3 dipolar and Diels-Alder cycloadditions, nucleophilic ring opening, non-aldol carbonyl chemistry, and additions to carbon-carbon multiple bonds. Cu(I) catalyzed versions of the Huisgen acetylene-azide cycloaddition, also known as azide click reaction, became the gold standard of click chemistry and have been applied in fields ranging from material science to chemical biology and drug development. However, the use of cytotoxic Cu (I) catalysts has largely precluded application of this click reaction in living systems. Recently discovered catalyst-free 1,3-dipolar cycloaddition of azides to cyclooctynes and dibenzocyclooctynes offers a bio-compatible version of the azide click reaction.

“Click” methods based on a Diels-Alder cycloaddition are also gaining popularity due to the fact that this reaction does not require catalysts, can proceed in high yield under physiological conditions, and does not produce any by-products. However, Diels-Alder cycloaddition reactions are often slow and require either thermal activation or the use of chemical promoters for the in situ generation of highly reactive dienes.

Thus, there remains a need for catalyst-free ligation methods for connection or immobilization of various compounds.

### SUMMARY

The present disclosure provides methods for labeling a substrate.

In one embodiment, the method includes: generating an o-quinone methide attached to a surface of a substrate and having the formula:

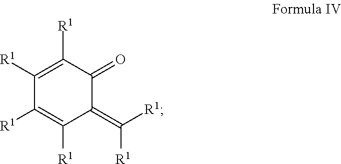

and contacting the o-quinone methide with a polarized olefin having the formula:

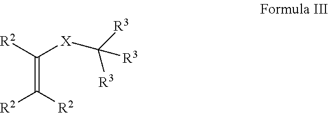

under conditions effective to form a hetero-Diels-Alder adduct, wherein: each R¹ is independently H, halogen, or an organic group, with the proviso that at least one R¹ includes a linker group attached to the surface of the substrate; each R² is independently H or an organic group; each R³ is independently H, halogen, or an organic group; and X is O or NR⁴, wherein R⁴ is H or an organic group, with the proviso that at least one R², R³, or R⁴ includes a detectable label. Optionally, R³ or R⁴ includes the detectable label, and the method can further include removing the detectable label from the surface of the substrate by hydrolyzing the C—X bond. Optionally, two or more R¹ groups may be combined to faun one or more rings (e.g. to form an o-naphthoquinone methide or an o-anthraquinone methide). Optionally, two or more R² groups may be combined to form one or more rings. Optionally, two or more R³ and/or R⁴ groups may be combined to faun one or more rings. In certain preferred embodiments, the o-quinone methide is an o-naphthoquinone methide having one of the formulas:

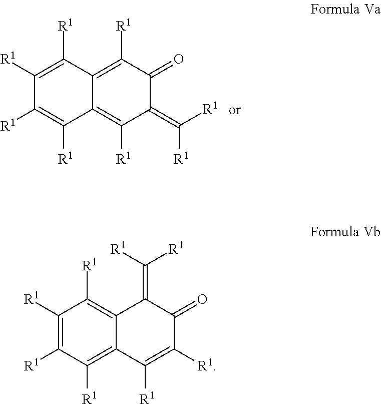

In another embodiment, the method includes: providing a precursor compound attached to a surface of a substrate and having the formula:

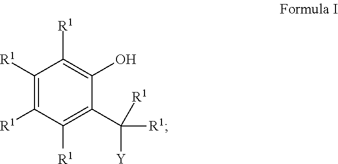

irradiating the precursor compound under conditions effective to form an o-quinone methide having the formula:

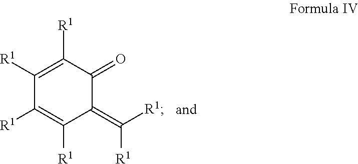

contacting the o-quinone methide with a compound having the formula

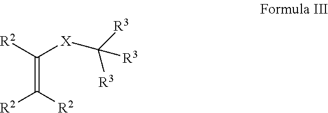

under conditions effective to form a hetero-Diels-Alder adduct, wherein: each R¹ is independently H, halogen, or an organic group, with the proviso that at least one R¹ includes a linker group attached to the surface of the substrate; each R² is independently H or an organic group; each R³ is independently H, halogen, or an organic, group; X is O or NR⁴; Y is OR⁵, NR⁵₂, NR⁵₃⁺ (Z_{1/q})⁻ wherein Z is an anion having a negative charge of q; and each R⁴ and R⁵ is independently H or an organic group, with the proviso that at least one R², R³, or R⁴ includes a detectable label. Optionally, two or more R¹ groups may be combined to form one or more rings (e.g. to form a naphthalene ring in a precursor of an o-naphthoquinone methide; or to form an anthracene ring in a precursor of an o-anthraquinone methide). Optionally, two or more R² groups may be combined to form one or more rings. Optionally, two or more R³ and/or R⁴ groups may be combined to faun one or more rings. Optionally, two or more R⁵ groups may be combined to form one or more rings. Optionally, the precursor compound can be irradiated in the presence of the polarized olefin. In certain embodiments, irradiating the precursor compound can include pattern-wise irradiating the substrate to provide a pattern-wise labeled surface of the substrate. In certain preferred embodiments, the precursor compound has one of the formulas:

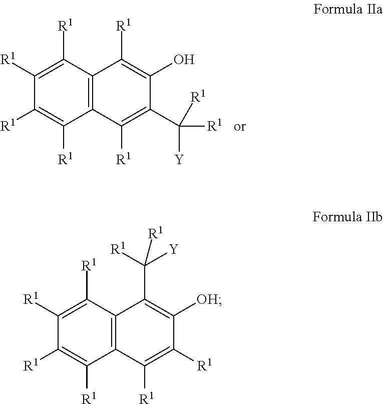

and irradiating the precursor compound under conditions effective to form an o-quinone methide forms an o-naphthoquinone methide having one of the formulas:

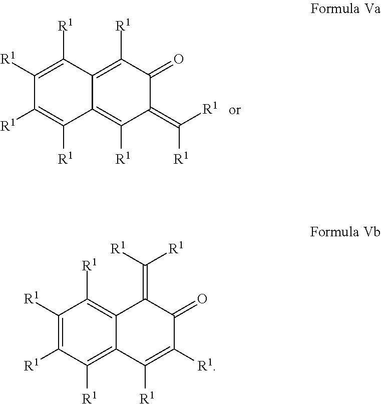

In another embodiment, the method includes: generating an o-quinone methide having the formula:

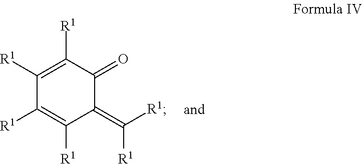

contacting the o-quinone methide with a polarized olefin attached to a surface of a substrate and having the formula:

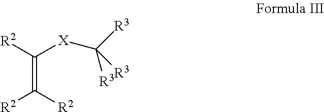

under conditions effective to form a hetero-Diels-Alder adduct, wherein: each R¹ is independently H, halogen, or an organic group, with the proviso that at least one R¹ includes a detectable label; each R² is independently H or an organic group; each R³ is independently H, halogen, or an organic group; and X is O or NR⁴, wherein R⁴ is H or an organic group, with the proviso that at least one R², R³, or R⁴ includes a linker group attached to the surface of the substrate. Optionally, two or more R¹ groups may be combined to foam one or more rings (e.g. to form an o-naphthoquinone methide or an o-anthraquinone methide). Optionally, two or more R² groups may be combined to faun one or more rings. Optionally, two or more R³ and/or R⁴ groups may be combined to form one or more rings. Optionally, the method can further include removing the detectable label from the surface of the substrate by hydrolyzing the C—X bond. In certain preferred embodiments, the o-quinone methide is an o-naphthoquinone methide having one of the formulas:

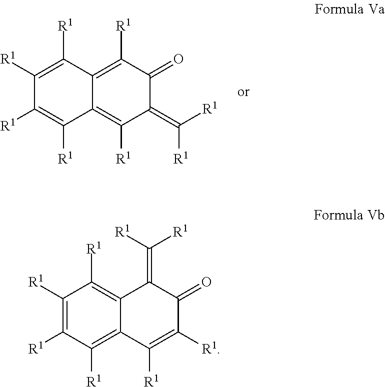

In another embodiment, the method includes: providing a precursor compound having the formula:

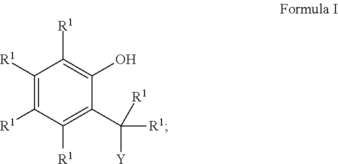

irradiating the precursor compound under conditions effective to form an o-quinone methide having the formula:

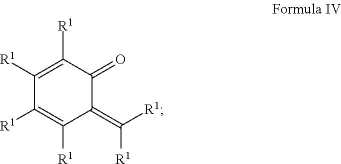

and contacting the o-quinone methide with a polarized olefin attached to a surface of a substrate and having the formula:

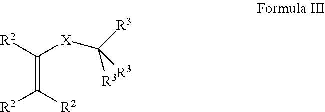

under conditions effective to form a hetero-Diels-Alder adduct, wherein: each R¹ is independently H, halogen, or an organic group, with the proviso that at least one R¹ includes a detectable label; each R² is independently H or an organic group; each R³ is independently H, halogen, or an organic group; X is O or NR⁴; Y is OR⁵, NR⁵₂, NR⁵₃⁺ (Zu_{q})⁻ wherein Z is an anion having a negative charge of q; and each R⁴ and R⁵ is independently H or an organic group, with the proviso that at least one R², R³, or R⁴ includes a linker group attached to the surface of the substrate. Optionally, two or more R¹ groups may be combined to form one or more rings (e.g. to form a naphthalene ring in a precursor of an o-naphthoquinone methide; or to form an anthracene ring in a precursor of an o-anthraquinone methide). Optionally, two or more R² groups may be combined to form one or more rings. Optionally, two or more R³ and/or R⁴ groups may be combined to form one or more rings. Optionally, two or more R⁵ groups may be combined to fowl one or more rings. Optionally, the precursor compound can be irradiated in the presence of the polarized olefin. Optionally, irradiating the precursor compound can include pattern-wise irradiating the substrate to provide a pattern-wise labeled surface of the substrate. In certain preferred embodiments, the precursor compound has one of the formulas:

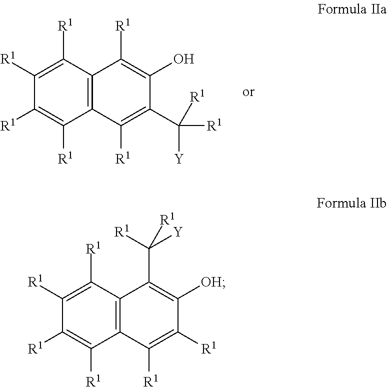

and irradiating the precursor compound under conditions effective to form an o-quinone methide forms an o-naphthoquinone methide having one of the formulas:

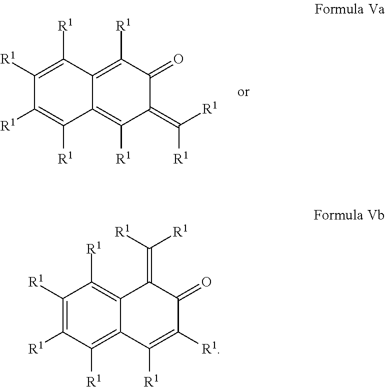

In another aspect, the present disclosure provides a precursor compound having the formula:

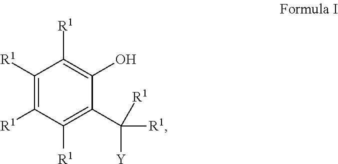

wherein: each R¹ is independently H, halogen, or an organic group; Y is OR⁵, NR⁵₂, NR⁵₃⁺ (Z_{1/q})⁻ wherein Z is an anion having a negative charge of q; and each R⁴ and R⁵ is independently H or an organic group, with the proviso that at least one R¹ includes a detectable label or a linker group attached to a surface of a substrate. Optionally, two or more R¹ groups may be combined to form one or more rings (e.g. to form a naphthalene ring or an anthracene ring). Optionally, two or more R⁵ groups may be combined to faun one or more rings. In certain preferred embodiments, the precursor compound has one of the formulas:

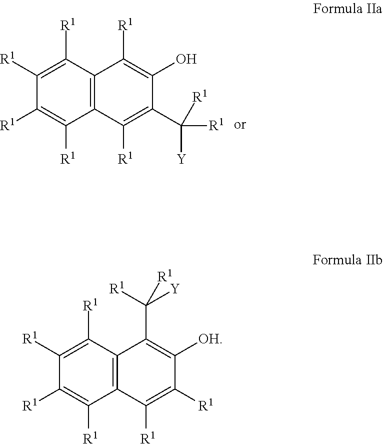

In another aspect, the present disclosure provides an o-quinone methide having the formula:

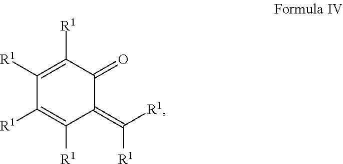

wherein each R¹ is independently H, halogen, or an organic group, with the proviso that at least one R¹ includes a detectable label or a linker group attached to a surface of a substrate. Optionally, two or more R¹ groups may be combined to form one or more rings (e.g. to form an o-naphthoquinone methide or an o-anthraquinone methide). In certain preferred embodiments, the o-quinone methide is an o-naphthoquinone methide of one of the formulas:

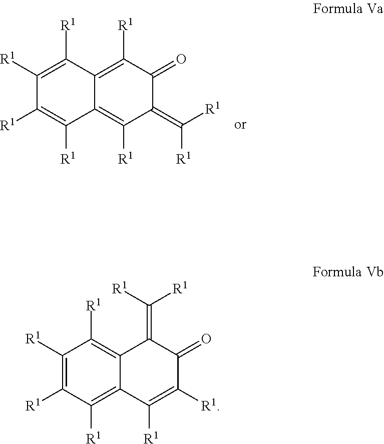

The reactions recited in the present disclosure can allow for the development of reagentless and catalyst-free ligation methods. In some embodiments, these methods are based on the in situ photochemical generation of the reactive component of a cycloaddition reaction. This approach can also expand the utility of “click” techniques by permitting temporal and spatial (potentially even 3-D) control over the process. Photogenerated click-substrates are expected to cover a broad range of reactivities from 0.1 to 10⁴ M⁻¹ s⁻¹. The advantages of photo-triggered click approaches to ligation and immobilization are well recognized.

Photochemical immobilization of carbohydrates, proteins, DNA fragments, antibodies, and other substrates allows for the formation of patterned or gradient arrays on various surfaces. These techniques can be used in the development of novel high throughput analytical methods.

The photo-triggered click reactions disclosed herein can expand the utility of this technique. First, the photoreactions employed can produce reactive components that have higher quantum and quantitative chemical yields. As a result, methods described herein typically require only short irradiation with a low intensity lamp, thus exhibiting much less light-induced toxicity in cells. Second, photochemical hetero-Diels-Alder reactions are very fast and allow for high spatial resolution of labeling or ligation. In addition, they provide a ligation method orthogonal to the azide click reaction. The o-quinone methides do react with water, but this reaction can actually be beneficial, because it regenerates the precursor compound. Thus, photo-ligation methods disclosed herein can be compatible with biological media.

### DEFINITIONS

As used herein, “a,” “an,” “the,” and “at least one” are used interchangeably and mean one or more than one.

As used herein, the term “comprising,” which is synonymous with “including” or “containing,” is inclusive, open-ended, and does not exclude additional unrecited elements or method steps.

The above brief description of various embodiments of the present invention is not intended to describe each embodiment or every implementation of the present invention. Rather, a more complete understanding of the invention will become apparent and appreciated by reference to the following description and claims in view of the accompanying drawings. Further, it is to be understood that other embodiments may be utilized and structural changes may be made without departing from the scope of the present invention.

### BRIEF DESCRIPTION OF THE DRAWINGS

FIGS. 1 and 2 are schematic illustrations of embodiments of the generation of an o-naphthoquinone methide by irradiation of a precursor compound, followed by a hetero-Diels-Alder reaction with a polarized olefin to form a hetero-Diels-Alder adduct.

FIG. 3 illustrates the ultraviolet (UV)-spectra of approximately 10⁻⁴ M solutions of 3-hydroxy-2-naphthalenemethanol (2a) and adduct 5a in (1:1) acetonitrile-water mixture.

FIG. 4 is a schematic illustration of embodiments of the generation of an o-naphthoquinone methide by irradiation of a precursor compound, followed by trapping of the o-naphthoquinone methide by methyl thiol.

FIG. 5 is a schematic illustration of the hydrolysis of an exemplary hetero-Diels-Alder adduct under acidic conditions.

FIGS. 6 and 7 are schematic illustrations of embodiments of the generation of an o-naphthoquinone methide by irradiation of a precursor compound, followed by a selective hetero-Diels-Alder reaction with polarized olefins to form hetero-Diels-Alder adducts.

FIG. 8 is a schematic illustration of the hydrolysis of an exemplary hetero-Diels-Alder adduct.

FIGS. 9, 10, and 11 are schematic illustrations of embodiments of the generation of an o-naphthoquinone methide by irradiation of a precursor compound, followed by a hetero-Diels-Alder reaction with polarized olefins to form hetero-Diels-Alder adducts.

FIG. 12 is a schematic illustration for an exemplary method for preparing an embodiment of a precursor compound. Reagents and conditions: (a) K₂CO₃, acetone, reflux, 70%; (b) LiAlH₄, tetrahydrofuran (THF), 92%.

FIGS. 13(a) and (b) are schematic illustrations for an exemplary method for preparing embodiment of polarized olefins.

FIG. 14 is a schematic illustration for an exemplary method for preparing an embodiment of a precursor compound. Reagents and conditions: (a) K₂CO₃, acetone, reflux, 65%; (b) LiAlH₄, THF, 92%; then aqueous HF/CH₃CN, 85% over 2 steps; (c) TsOH, 2,2-dimethoxypropane, acetone, 90%; (d) TsCI, pyridine, DCM, 82%; (e) NaN₃, DMF, 60° C., 80%; (f) LiAlH₄, Et₂O, 85%; (g) EDC, DMAP, DMF, 83%; (h) Amberlyst-15, methanol, room temperature, 95%.

FIG. 15 illustrates the ultraviolet (UV)-spectra of the biotin-3-hydroxy-2-naphthalenemethanol conjugate 2d.

FIG. 16 is a schematic illustration of an embodiment of the generation of an o-naphthoquinone methide by irradiation of a precursor compound, followed by a hetero-Diels-Alder reaction with a polarized olefin to faun a hetero-Diels-Alder adduct.

FIG. 17 is a schematic representation of an embodiment of the preparation of vinyl ether functionalized surface of a substrate (e.g., glass slides).

FIG. 18 illustrates embodiments showing FITC-Avidin stained biotinylated glass slides: (a) comparison of blank (vinyl ether slide) and dark reaction slide (vinyl ether slide incubated in aqueous biotin-conjugate solution in the dark) with flood irradiated slide (b) Biotinylated glass slides with different degree of functionalization (3-10 minutes photolysis) (c) Biotinylated slide (10 minutes sample) after 30 minutes exposure to 300 nm light in aqueous phosphate solution.

FIG. 19 illustrates embodiments showing FITC-Avidin stained biotinylated glass slides: (a) comparison of blank (vinyl ether slide) and dark reaction slide (vinyl ether slide incubated in aqueous biotin-conjugate solution in the dark) with flood irradiated slide (b) Biotinylated glass slides with different degree of functionalization (3-10 minutes photolysis) (c) Biotinylated slide (10 minutes sample) after 30 minutes exposure to 300 nm light in aqueous phosphate solution.

FIG. 20 illustrates embodiments showing water contact angles of functionalized: (a) vinyl ether glass slide; (b) vinyl ether glass slide after photo-click reaction 2a; (c) vinyl ether glass slide after photo-click reaction 2b; and (d) vinyl ether glass slide after photo-click reaction 2d.

FIG. 21 illustrates Fourier transform infrared (FTIR) spectra for embodiments of functionalized glass surfaces: (a) vinyl ether glass slide after photo-click reaction 2c (b) vinyl ether glass slide after photo-click reaction 2a (c) vinyl ether glass slide.

FIG. 22 illustrates embodiments showing patterned surface derivatization: (a) by prepositioning 2 μL drops of photoactive click reagents on the vinyl ether glass slide (b1) by placing the vinyl ether glass slide and irradiating through “UGA” mask (original scan) (b2) enlarged version of the original scan b1.

FIG. 23 illustrates embodiments showing patterned surface derivatization: by prepositioning 2 μL drops of the photoactive click reagent on the vinyl ether glass slide (a) washed for 1 hour without PEGlation before FITC-Avidin staining (b) washed for 6 hours without PEGlation before FITC-Avidin staining (c) washed for 16 hours without PEGlation before FITC-Avidin staining (d) washed for 1 hour with PEGlation before FITC-Avidin staining.

FIG. 24 is an illustration of UV-spectra of approximately 10⁴ M solutions of 2-hydroxynaphthochroman (15f, dash line) and the solution 15f released by the photo-clicked surface upon dilute acid (0.1M) treatment (solid line) in (1:1) acetonitrile-water mixture.

FIG. 25 is a schematic illustration of embodiments of the generation of an o-naphthoquinone methide by irradiation of a precursor compound, followed by a hetero-Diels-Alder reaction with a polarized olefin to form a hetero-Diels-Alder adduct; and hydrolysis of the exemplary hetero-Diels-Alder adduct under acidic conditions.

FIG. 26 illustrates embodiments showing FITC-Avidin stained biotinylated glass slides: (a1) unhydrolyzed glass slide functionalized through β-carbon atom of vinyl ether (a2) hydrolyzed glass slide functionalized through β-carbon atom of vinyl ether (b1) unhydrolyzed glass slide functionalized through oxygen atom of vinyl ether (b2) hydrolyzed glass slide functionalized through oxygen atom of vinyl ether.

### DETAILED DESCRIPTION OF ILLUSTRATIVE EMBODIMENTS

The present disclosure provides methods for labeling a substrate using a hetero-Diels-Alder reaction. The hetero-Diels-Alder reaction includes the reaction of an o-quinone methide (e.g., an o-naphthoquinone methide) with a polarized olefin to a hetero-Diels-Alder adduct. The o-quinone methide or the polarized olefin can be attached to a surface of a substrate, and the other of the o-quinone methide or the polarized olefin can include a detectable label. The o-quinone methide can conveniently be generated by irradiation of a precursor compound, preferably in an aqueous solution, suspension, or dispersion.

### Precursor Compounds.

In one embodiment, an o-quinone methide is generated from a precursor compound having the formula:

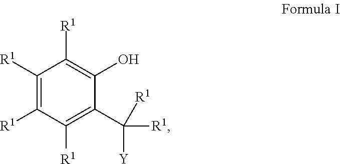

wherein: each R¹ is independently H, halogen, or an organic group; Y is OR⁵, NR⁵₂, NR⁵₃⁺ (Z_{1/q})⁻ wherein Z is an anion having a negative charge of q; and each R⁴ and R⁵ is independently H or an organic group, with the proviso that at least one R¹ includes a detectable label or a linker group attached to a surface of a substrate. Optionally, two or more R¹ groups may be combined to form one or more rings (e.g. to form a naphthalene ring or an anthracene ring). Optionally, two or more R⁵ groups may be combined to form one or more rings. In certain preferred embodiments, an o-naphthoquinone methide is generated from a precursor compound having one of the formulas:

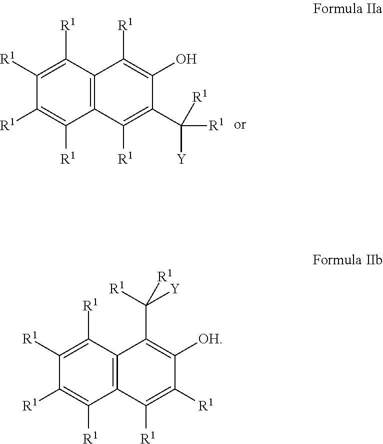

When R¹ and/or R⁵ represent an organic group, preferably the organic group is a carbon-bound (i.e., the bond to the group is to a carbon atom of the organic group) organic group. In certain embodiments, the organic group is an aliphatic group such as a C1-C20 aliphatic group, in some embodiments a C1-C10 aliphatic group, and in some embodiments a C1-C10 hydrocarbon moiety.

As used herein, the term “organic group” is used for the purpose of this disclosure to mean a hydrocarbon group that is classified as an aliphatic group, cyclic group, or combination of aliphatic and cyclic groups (e.g., alkaryl and aralkyl groups). In the context of the present disclosure, suitable organic groups for hetero-Diels-Alder reactants or precursors thereof, as described herein, are those that do not interfere with a light-induced photodehydration reaction and/or a [4+2] cycloaddition reaction. In the context of the present disclosure, the term “aliphatic group” means a saturated or unsaturated linear or branched hydrocarbon group. This term is used to encompass alkyl, alkenyl, and alkynyl groups, for example. The term “alkyl group” means a saturated linear or branched monovalent hydrocarbon group including, for example, methyl, ethyl, n-propyl, isopropyl, tert-butyl, amyl, heptyl, and the like. The term “alkenyl group” means an unsaturated, linear or branched monovalent hydrocarbon group with one or more olefinically unsaturated groups (i.e., carbon-carbon double bonds), such as a vinyl group. The term “alkynyl group” means an unsaturated, linear or branched monovalent hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polynuclear aromatic hydrocarbon group. The term “heterocyclic group” means a closed ring hydrocarbon in which one or more of the atoms in the ring is an element other than carbon (e.g., nitrogen, oxygen, sulfur, etc.).

As a means of simplifying the discussion and the recitation of certain terminology used throughout this application, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoallyls, etc. On the other hand, the phrase “alkyl moiety” is limited to the inclusion of only pure open chain saturated hydrocarbon alkyl substituents, such as methyl, ethyl, propyl, tert-butyl, and the like.

### Substrates.

In some embodiments, the precursor compound, the o-quinone methide (e.g., an o-naphthoquinone methide), the polarized olefin, and/or the hetero-Diels-Alder adduct are attached to the surface of a substrate. A substrate can be of any suitable size and/or shape. For example, useful substrates include planar surfaces and beads (e.g., particles such a microbeads having an average size less than 0.1 mm). Substrates including a wide variety of materials can be used. For example, suitable substrates can include glass, quartz, silica, metal, semi-conductor, polymer, membrane, liposome, micelle, macromolecule, a biomaterial, or a combination thereof. As used herein, the term “biomaterial” is meant to include any biological material or material that can be used in a biological method or application. Such materials include, but are not limited to cells, viruses, small multicellular organisms, DNA, RNA, peptides, polypeptides, proteins, carbohydrates, lipids, tissues, or combinations thereof.

### Linkers

For embodiments in which the precursor compound, the o-quinone methide (e.g., an o-naphthoquinone methide), the polarized olefin, and/or the hetero-Diels-Alder adduct are attached to the surface of a substrate, the attachment may include a linker. A wide variety of linkers known to those of skill in the art can be used to attach the substrate. For example, useful linkers can include, but are not limited to, aliphatic chains and alkylene glycol oligomers (e.g., ethylene glycol oligomers).

### Detectable Labels

In some embodiments, a detectable label is attached to the precursor compound, the o-quinone methide (e.g., an o-naphthoquinone methide), the polarized olefin, and/or the hetero-Diels-Alder adduct. As used herein, a detectable label is meant to include any group or functionality desired that can be detected before and/or after attachment to the surface of a substrate. The labels can be detected by a wide variety of convenient methods including, but not limited to, fluorescence, phosphorescence, radiation detection, optical and electrochemical methods, surface plasmon resonance imaging (SPR¹), or combinations thereof.

In some embodiments, the detectable label can include a probe molecule. A wide variety of probe molecules can be used including, for example, DNA, peptides, polypeptides, proteins, and combinations thereof.

Generation of o-quinone methide.

Precursor compounds as disclosed herein can be irradiated to generate o-quinone methides (e.g., o-naphthoquinone methides). Typically, the precursor compound is irradiated in an aqueous solution, suspension, or dispersion. As used herein, an aqueous solution, suspension, or dispersion is intended to include liquids that include, but are not limited to, water. Thus, aqueous liquids can also include, for example, organic solvents such as acetonitrile.

Typically, the aqueous solution, suspension, or dispersion of the precursor compound is irradiated at a wavelength of 250 nm to 350 nm. Convenient wavelengths include, for example, 350 nm such as those available from a fluorescent UV lamp. Other convenient wavelengths include, for example, 266 nm and 355 nm. Typically, the aqueous solution, suspension, or dispersion of the precursor compound is irradiated under ambient conditions for a time sufficient for the desired reactions to occur. It would be clear to one of skill in the art that suitable irradiation times can be varied depending on a number of factors such as intensity of the irradiation and the area or volume being irradiated. An exemplary suitable time for the irradiation can be 0.5 minutes to 5 minutes.

Conveniently, the precursor compound can be irradiated in the presence of a polarized olefin, which can react with generated o-quinone methide (e.g., an o-naphthoquinone methide) in a hetero-Diels-Alder reaction. For embodiments in which the precursor compound is irradiated in the presence of the polarized olefin, the substrate (i.e., the surface of which is attached to the precursor compound or the polarized olefin) can be pattern-wise irradiated to provide a pattern-wise labeled surface of the substrate.

o-Quinone methide.

Irradiation of the precursor compound can generate an o-quinone methide having the formula:

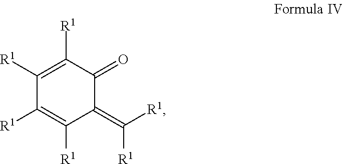

wherein each R¹ is independently H, halogen, or an organic group, with the proviso that at least one R¹ includes a detectable label or a linker group attached to a surface of a substrate. Optionally, two or more R¹ groups may be combined to form one or more rings (e.g. to form an o-naphthoquinone methide or an o-anthraquinone methide). In certain preferred embodiments, the o-quinone methide is an o-naphthoquinone having one of the formulas:

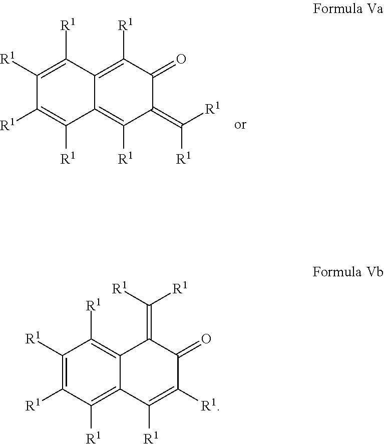

When R¹ represents an organic group, preferably the organic group is a carbon-bound (i.e., the bond to the group is to a carbon atom of the organic group) organic group. In certain embodiments, the organic group is an aliphatic group such as a C1-C20 aliphatic group, in some embodiments a C1-C10 aliphatic group, and in some embodiments a C1-C10 hydrocarbon moiety.

### Polarized Olefin

The o-naphthoquinone methide can react with a polarized olefin in a hetero-Diels-Alder reaction. Suitable polarized olefins are, for example, of the formula:

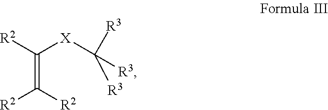

wherein each R² is independently H or an organic group; each R³ is independently H, halogen, or an organic group; and X is O or NR⁴, wherein R⁴ is H or an organic group. In some embodiments, at least one R², R³, or R⁴ includes a linker group attached to the surface of the substrate. In some other embodiments, at least one R², R³, or R⁴ includes a detectable label.

When R², R³, or R⁴ represents an organic group, preferably the organic group is a carbon-bound (i.e., the bond to the group is to a carbon atom of the organic group) organic group. In certain embodiments, the organic group is an aliphatic group such as a C1-C20 aliphatic group, in some embodiments a C1-C10 aliphatic group, and in some embodiments a C1-C10 hydrocarbon moiety.

### Hetero-Diels-Alder Adduct

The o-quinone methide disclosed herein can react with a polarized olefin in a hetero-Diels-Alder reaction. Conditions effective for the hetero-Diels-Alder reaction include contacting the o-quinone methide (e.g., an o-naphthoquinone methide) and the polarized olefin in an aqueous solution, suspension, or dispersion, typically under ambient conditions. As used herein, an aqueous solution, suspension, or dispersion is intended to include liquids that include, but are not limited to, water. Thus, aqueous liquids can also include, for example, organic solvents such as acetonitrile.

A hetero-Diels-Alder reaction of the o-quinone methide disclosed herein with a polarized olefin can form an adduct having the formula:

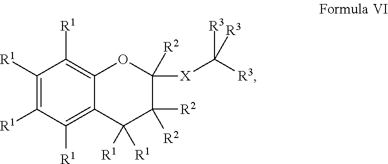

wherein each R¹ is independently H, halogen, or an organic group; each R² is independently H or an organic group; each R³ is independently H, halogen, or an organic group; and X is O or NR⁴, wherein R⁴ is H or an organic group. Optionally, two or more R¹ groups may be combined to form one or more rings. Optionally, two or more R² groups may be combined to form one or more rings. Optionally, two or more R³ and/or R⁴ groups may be combined to form one or more rings. In certain preferred embodiments, the hetero-Diels-Alder reaction of an o-naphthoquinone methide disclosed herein with a polarized olefin can form an adduct of one of the formulas:

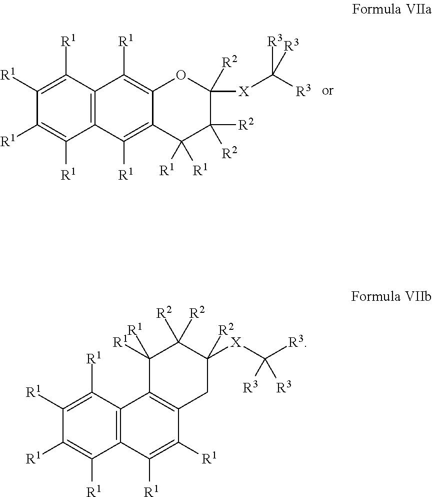

When R¹, R², R³, or R⁴ represents an organic group, preferably the organic group is a carbon-bound (i.e., the bond to the group is to a carbon atom of the organic group) organic group. In certain embodiments, the organic group is an aliphatic group such as a C1-C20 aliphatic group, in some embodiments a C1-C10 aliphatic group, and in some embodiments a C1-C10 hydrocarbon moiety.

In some embodiments, at least one R¹ includes a linker group attached to the surface of the substrate, and at least one R², R³, or R⁴ includes a detectable label, thus providing a label attached to the surface of the substrate. For embodiments in which R³ or R⁴ includes the detectable label, the detectable label can optionally be removed from the surface of the substrate by hydrolyzing the C—X bond.

In some other embodiments, at least one R¹ includes a detectable label and at least one R², R³, or R⁴ includes a linker group attached to the surface of the substrate, thus providing a label attached to the surface of the substrate. The detectable label can optionally be removed from the surface of the substrate by hydrolyzing the C—X bond.

The following examples are offered to further illustrate various specific embodiments and techniques of the present disclosure. It should be understood, however, that many variations and modifications understood by those of ordinary skill in the art may be made while remaining within the scope of the present disclosure. Therefore, the scope of the disclosure is not intended to be limited by the following examples.

### Example 1

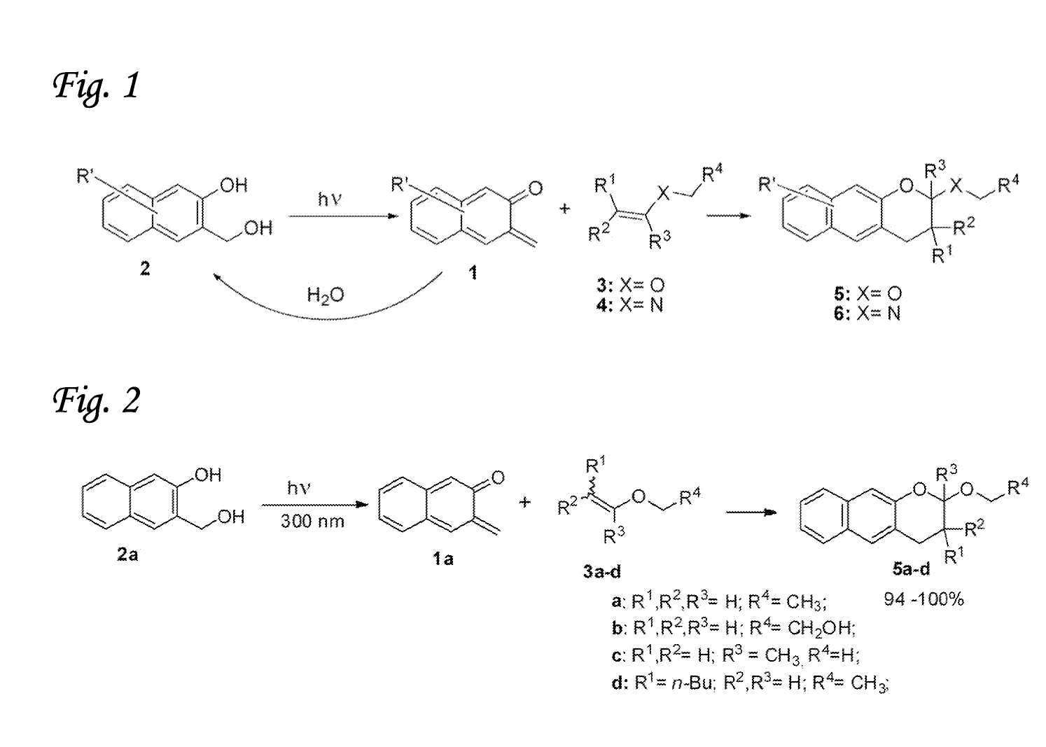

The light-induced click reaction for ligation of various molecules is based on the photochemical generation of o-napthoquinone methides in aqueous solution from 3-hydroxy-2-naphthalenemethanol precursor. The naphthyl chromophore allows for the activation using longer wavelength light (300-350 nm) and holds advantage over o-benzoquinone methide precursors such as o-hydroxybenzyl alcohol. Irradiation of 3-hydroxy-2-naphthalenemethanol chromophore (2) results in efficient dehydration of the substrate and the formation of o-naphthoquinone methide (oNQM) 1 (FIG. 1). In the presence of vinyl ethers (3) or enamines (4), oNQMs undergo very rapid Diels-Alder cycloaddition to yield substituted 2-alkoxy-3,4-dihydro-2H-naphtho[2,3-b]pyran (5) or 2-alkylamino-3,4-dihydro-2H-naphtho[2,3-b]pyran (6) as shown in FIG. 1. Various substituents can be introduced in aromatic rings of 2 and in vinyl component (3, 4) to serve as linker to substrates of interest (FIG. 1). In the absence of vinyl ethers, enamines, or other trapping agents o-quinone methides add water to regenerate the starting material.

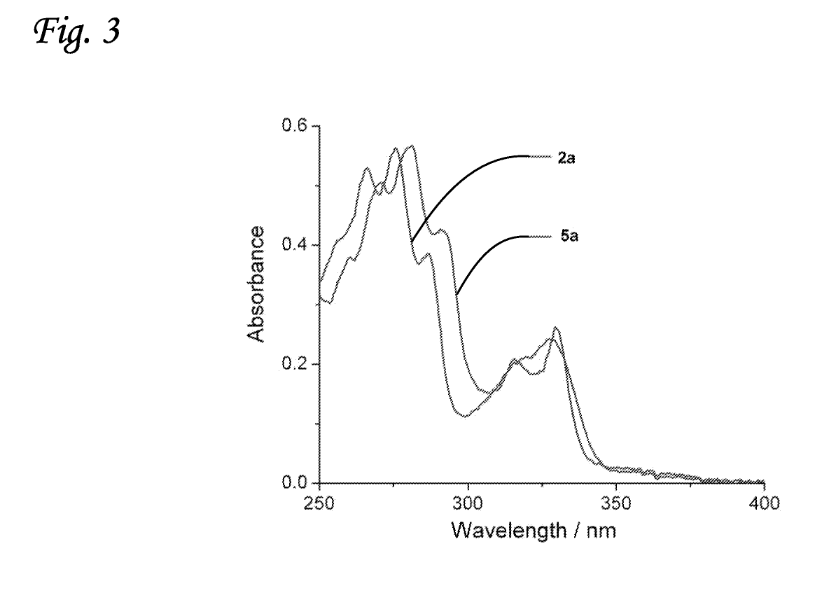

Photophysical properties, generation, and reactivity of oNQMs are discussed on the example of 3-hydroxy-2-naphthalenemethanol 2a (R′═H, FIG. 2). UV spectra of 3-hydroxy-2-naphthalenemethanol (2a) resemble the spectrum of 2-naphthol and contains two major absorption bands above 210 nm: at λₘₐₓ 275 nm (log ε=4.06 and at λₘₐₓ=324 nm (log ε=3.70, FIG. 3). Both bands show pronounced vibrational structure. 2a also show strong fluorescence with quantum yield of Φ_{FI}=0.230±0.002 and generates the quinine methide with quantum yield 0.20±0.2. The emission spectra of 2a in aqueous solutions contain two major bands at 360 nm and 423 nm. The fluorescent life-time is τ_{FL}˜7 ns. While the emission spectra and fluorescent quantum yields of 2a resemble that of 2-naphthol (Φ_{FI}=0.27) (J. B. Birks, Photophysics of Aromatic Molecules. Wiley Interscience, New York, 1970, 703 p; Datta et al., Spec. Acta A, 2006, 64, 11), the fluorescence life-time is somewhat shorter than for 2-naphthol (τ_{FL}˜11 ns) (Wang et al., J. Org. Chem. 2002, 67, pp 2429; Berlman, I. B. Handbook of Fluorescence Spectra of Aromatic Molecules. Academic Press, New York, 1971, 473p). This observation apparently indicates that a new pathway for the excited state decay is opened for 2a.

Irradiation at 300 nm of 3-hydroxy-2-naphthalenemethanol (2a) results in the generation of oNQM 1a, which undergoes rapid hetero-Diels-Alder addition to ethyl vinyl ether (3a) to produce quantitative yield of 2-ethoxynaphthochroman 5a. The bimolecular rate of this reaction in aqueous solution was measured to be 4.07×10⁴ M⁻¹ s⁻¹. (Arumugam et al., J. Am. Chem. Soc. 2009, 131, 11892). In the absence of vinyl ether moieties, oNQM undergoes very efficient re-hydration (τ_{H2O} (1a)=7.4 ms) to generate the starting material and no detectable new products are observed. It is important to note that high yield of hetero-Diels-Alder adducts is achieved despite the presence of more than a thousand-fold excess of a nucleophilic solvent. This observation indicates that addition of ethyl vinyl ether to oNQMs is at least two orders of magnitude faster than a hydration reaction. The hetero-Diels-Alder adducts are formed almost quantitatively even at the low conversion. In order to check the utility of the photo click for the biological application, the photochemistry was also investigated using 350 nm wavelength light. The reaction is clean at 350 nm as observed in the case of 300 nm photolysis. The only difference is the extinction coefficient of the chromophore at 350 nm is relatively less than that at 300 nm and therefore it either requires higher concentration of the oNQM precursor or longer irradiation time to obtain similar chemical yields.

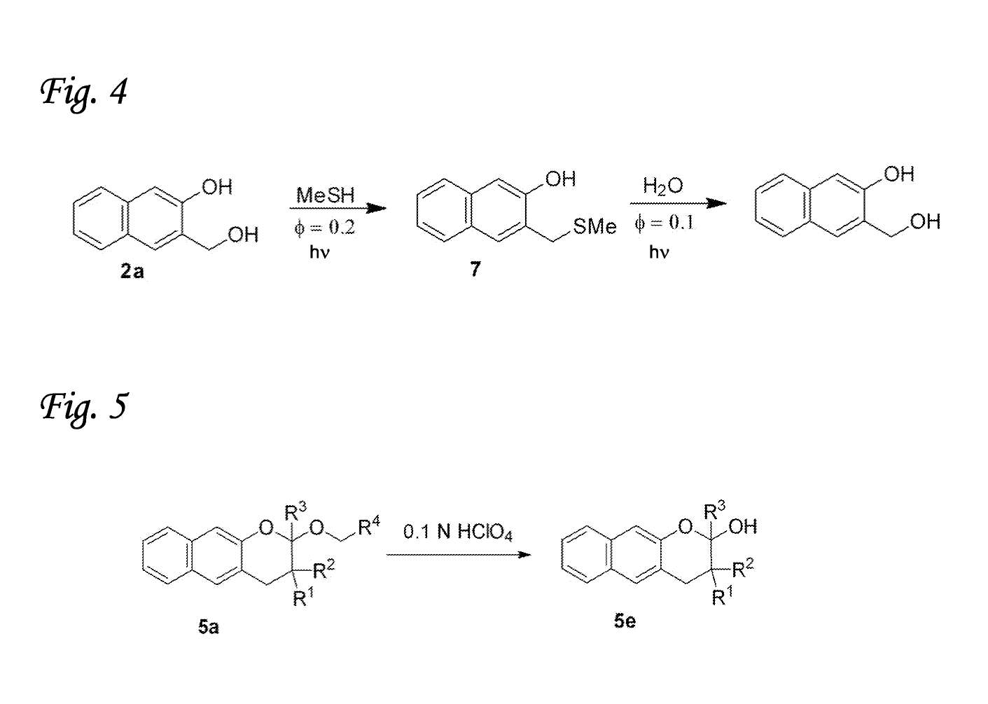

The oNQMs can also be can be intercepted by reactive nucleophiles such as azide anion and thiols. Our previous report on the kinetics of oNQM clearly demonstrates that the second order rate constant of thiol addition with oNQM is ˜1×10⁵M⁻¹ s⁻¹ (Arumugam et al., J. Am. Chem. Soc. 2009, 131, 11892), which is twice as that of hetero-Diels-Alder reaction with vinyl ethers. The thiol ether thus formed is equally stable in both acidic and basic condition. Recently, this property had been utilized for quinone methide based protein affinity labeling. (Jiang et al., ChemBioChem. 2009, 10, 635). However, the thiol ether is photo active and regenerates the oNQM that can undergo hydration to yield the diol as shown in FIG. 4. Though our quantum yield experiments on methyl thio ether analogue of 3-hydroxy-2-naphthalenemethanol indicates that the efficiency of oNQM generation is relatively less compared to the photo-dehydration from the diol 2a, the yield of ligation will depend on the time of irradiation. Besides, the high nucleophilicity of thiols makes it less orthogonal in the presence of other electron deficient carbons.

On other hand, 2-ethoxynaphthochroman 5a is photochemically stable and shows no decomposition even after prolonged irradiation at 254, 300 nm or 350 nm. The stability of 5a was measured in aqueous solution at pH˜7, 0.1N aqueous sodium hydroxide solution, and 0.1N aqueous perchloric acid for the 24 h period. HPLC analysis indicates that there are no changes in the concentration of 5a in aqueous solution of neutral pH and 0.1N aqueous sodium hydroxide solution. This observation clearly demonstrates that 5a is stable under neutral or basic conditions. 5a undergoes slow hydrolysis (life time τ˜9.5 h) in aqueous solution of perchloric acid of pH 1 to produce 2-hydroxynapthochroman (5e, FIG. 5). Besides, vinyl ethers are stable at ambient conditions and do not react in the dark.

TABLE 1
Chemical yields of hetero-Diels-Alder adducts and the hydration
product (2a) upon photolysis of 3-(ethoxymethyl)-2-naphthalenol
2c in the presence of various olefins 8-12.^{a, b.}

| | | | Chemical yield | |
|---|---|---|---|---|
| | | | of the hetero- | |
| | Chemical | | Diels-Alder | |
| | yield of 2a | | adduct | |
| Olefins | (% conversion) | | (% Conversion) | |
| 3a | 0 | (15) | 97 ± 2 | (15) |
| | 0 | (>99) | 96 ± 2 | (>99) |
| 3a + 8-12 | 0 | (>99) | 97 ± 2 | (>99) |
| 8 | 97 ± 2 | (14) | 0 | (14) |
| | 71 ± 3 | (89) | 0 | (89) |
| 9 | 93 ± 2 | (14) | 0 | (14) |
| | 69 ± 3 | (87) | 0 | (87) |
| 10 | 94 ± 2 | (14) | 0 | (14) |
| | 73 ± 2 | (90) | 0 | (89) |
| 11 | 96 ± 2 | (14) | 0 | (14) |
| | 71 ± 2 | (92) | 0 | (89) |
| 12 | 95 ± 2 | (14) | 0 | (14) |
| | 71 ± 2 | (85) | 0 | (89) |

^{a}[Olefin] = 100 mM; [oNQM precursor] = 1 mM.
^{b}Irradiation wavelength = 300 nm.

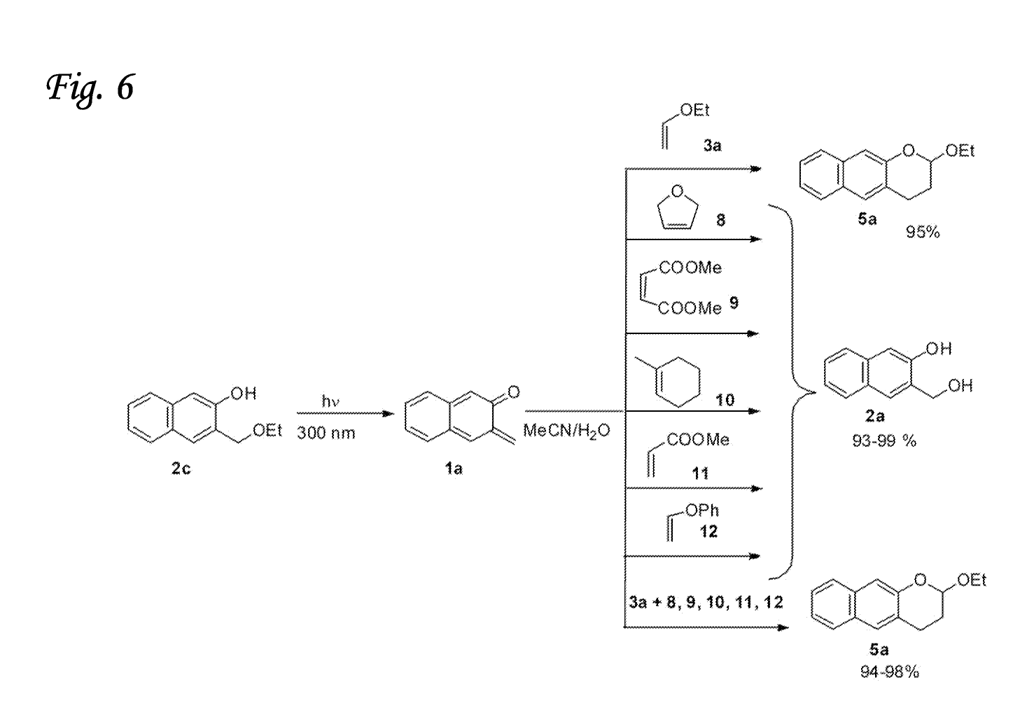

To examine the selectivity of the reaction of oNQM with various olefins, two set of experiments were carried out. First, 1 mM solution of oNQM precursor 2c was irradiated in the presence 0.1 M of ethyl vinyl ether (3a) and equal amounts (0.1 M each) of olefins 8-12 in 50% aqueous acetonitrile. HPLC analysis showed that only adduct 5a has been formed in excellent yields (FIG. 6) and the result are presented in Table 1. Among vinyl ethers, oNQM reacts only with aliphatic ether but does not form cycloaddition product with aromatic vinyl ether, such as 12.

Next, oNQM 1a was generated by 300 nm photolysis of 1 mM solutions of 3-(ethoxymethyl)-2-naphthalenol (2c) in 50% aqueous acetonitrile in the presence of 0.1 M of various olefins. The following olefins have been tested: ethyl vinyl ether (3a), 2,5-dihydrofuran (8), dimethyl maleate (9), 1-methylcyclohexene (10), methyl acrylate (11), and phenyl vinyl ether (12). Only in the presence of 3a, the formation of the quantitative yields of the hetero-Diels-Alder adduct 5a was observed both at lower and higher conversion. In all other cases, the only product of reaction was 3-hydroxy-2-naphthalenemethanol (2a), and no adducts of 1a to alkenes 8-12 were detected (FIG. 6). HPLC analysis reveals the quantitative formation of hydration product 2a in the latter cases (Table 1) at lower conversion. At higher conversion the yield of hydration product is some what reduced due to possible oligomerization of oNQM. However no cyclo-addition product is observed, which suggests that the hydration of 1a is much faster than reaction of 1a with olefins 8-12. Whereas, the hetero-Diels-Alder reaction adduct formation is the major reaction in the presence of 3a. These set of experiments clearly show that o-quinone methides are selective towards electron rich polarized alkenes, such as vinyl ether 3a.

TABLE 2
Rate of decay of 2, 3 NQM in the presence of various olefins (0.04M)
in 50% phosphate buffer + 50% MeCN aqueous solution.

| | Pseudo first order rate |
|---|---|
| Olefin | constant of 2, 3 NQM (s⁻¹) |
| 50% Phosphate Buffer + 50% | 119 ± 6 |
| 3a | 2703 ± 55 |
| 8 | 125 ± 9 |
| 9 | 118 ± 4 |
| 10 | 123 ± 7 |
| 11 | 121 ± 8 |

Also, The rate of disappearance of 1a in wholly aqueous solutions in the presence of 0.04 M of the following olefins: ethyl vinyl ether (3a), 2,5-dihydrofuran (8), dimethyl maleate (9), 1-methylcyclohexene (10) and methyl acrylate (11) were examined. It was found that only in the presence of 3a, the rate of decay was significantly enhanced (Table 2). In all other cases, the rate of decay was equal to the rate observed in neat water. This observation clearly indicates that only the reaction of oNQM with ethyl vinyl ether is able to compete with the hydration of oNQM. Whereas, in the case of non-polarized olefins and electron poor polarized olefins, the hydration reaction is the only reaction observed. This observation further confirms that o-quinone methides are very selective towards electron rich polarized olefin. Kinetic studies with olefin 12 can not be followed as it has substantial absorption at the excitation wavelength (266 nm). Also, oNQM readily reacts with the wide range of aliphatic vinyl ethers carrying various substituents at oxygen, α-carbon, and β-carbon atoms. For example, oNQM 1a was photochemically generated from 2a in the presence or 2-hydroxy ethyl vinyl ether (3b), isopropenyl methyl ether (3c), or 1-ethoxyhept-1-ene (3d). The corresponding photoproducts 5b-d were isolated in high yield and fully characterized. HPLC analysis of reaction mixtures confirmed nearly quantitative yields of adducts 5b-d in all three cases (Table 3).

TABLE 3
Chemical yield of the DA adduct upon photolysis 2a in the presence of
various vinyl ethers in 50% MeCN in 0.01N aqueous phosphate buffer
solution.^{a, b.}

| | Chemical yield of |
|---|---|
| Olefin | the DA adduct (% conversion) |
| 3a | 96 ± 2 (>99) |
| 3b | 94 ± 2 (>99) |
| 3c | 95 ± 3 (>99) |
| 3d | 97 ± 2 (>99) |

^{a}[Olefin] = 1.5 mM; [oNQM precursor] = 1 mM.
^{b}Irradiation wavelength = 300 nm.

TABLE 4
Rate of decay of 2, 3 NQM in the presence of various vinyl ethers
(0.05M) in 50% MeCN in 0.01N aqueous phosphate buffer solution.

| | Pseudo first order rate |
|---|---|
| Olefin | constant of 2, 3 NQM (s⁻¹) |
| 3a | 3100 ± 121 |
| 3b | 3050 ± 75 |
| 3c | 3080 ± 101 |
| 3d | 3090 ± 66 |

The rate of reaction of oNQM 1a with vinyl ethers 3b-d was determined using laser flash photolysis and was found to be very similar to that of ethyl vinyl ether 3a (Table 4). This observation indicates that variation of substitution at oxygen, α-carbon, and β-carbon atoms does not significantly affect the rate of the cycloaddition reaction.

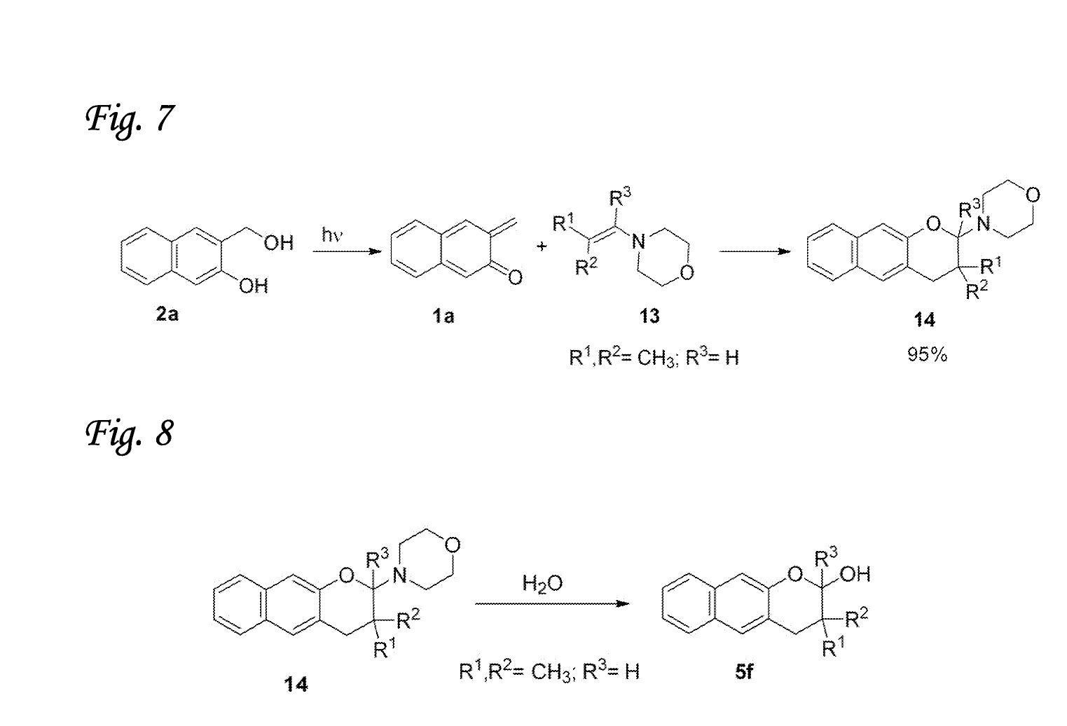

Photochemically-generated oNQM 1a also rapidly reacts with enamine 13 also results in quantitative formation of the adduct 14 (FIG. 7). In aqueous solutions adduct 14 undergoes rapid hydrolysis of the amino substituent to produce 3,4-dihydro-2-hydroxy-3,3-dimethyl-2H-Naphtho[2,3-b]pyran (5f, FIG. 8). The life-time of 14 at pH˜7 in wholly aqueous solution is τ=21.2±0.6 minutes.

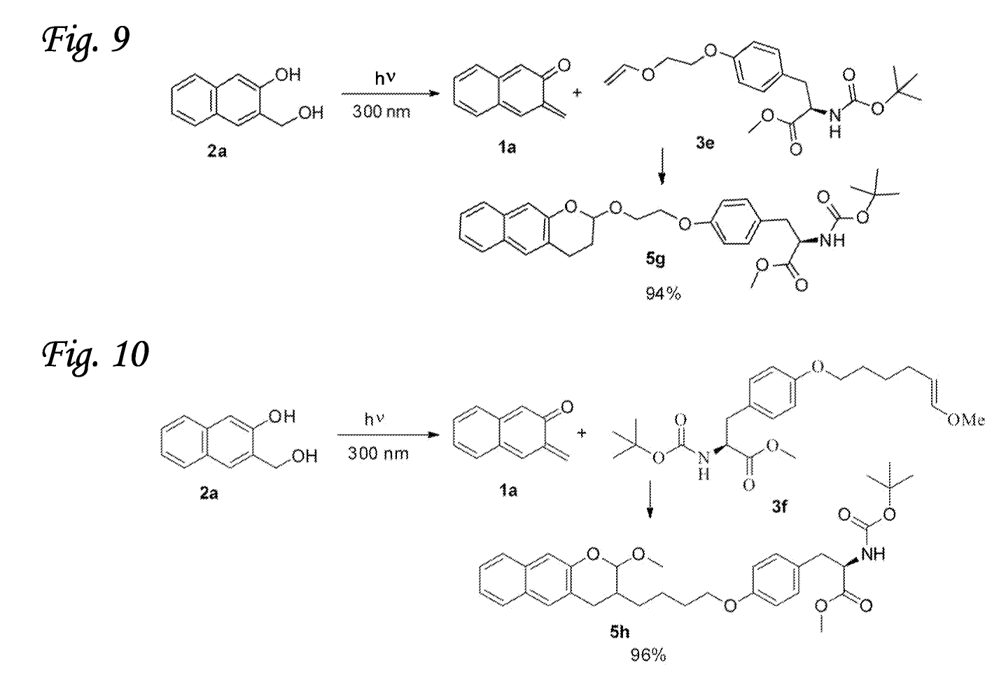

For the photo-ligation purposes the payload can be attached to the oxygen, α-carbon, and β-carbon atoms of the vinyl ether 3 (FIG. 2). For example, we have prepared vinyl ether 3e, which is conjugated to a protected amino acid tyrosine via ethylene glycol linker (FIG. 9). Irradiation of the precursor 2a in the presence of 3e resulted in the rapid and quantitative formation of the ligation product 5g (FIG. 9). The adduct 5g was isolated in 94% yield and fully characterized.

The strategy described above allows ligation of payloads through oxygen atom of vinyl ethers, which can be detached by acid hydrolysis at pH 1. For the application that requires permanent conjugation, the pay load is attached through β-carbon of vinyl ethers. For illustrative purpose, tyrosine was attached to β-carbon of the vinyl ether 3f through a short linker as shown in FIG. 10. As observed in the first approach, irradiation of the precursor 2a in the presence of the 1.5 equivalents of vinyl ether 3f resulted in the quantitative formation of the ligation product (FIG. 10). Thus it has been clearly demonstrated that vinyl ether system provides dual mode of ligation, one mode allows conjugation via acid labile C—O bond and another mode allows ligation via permanent C—C bond.

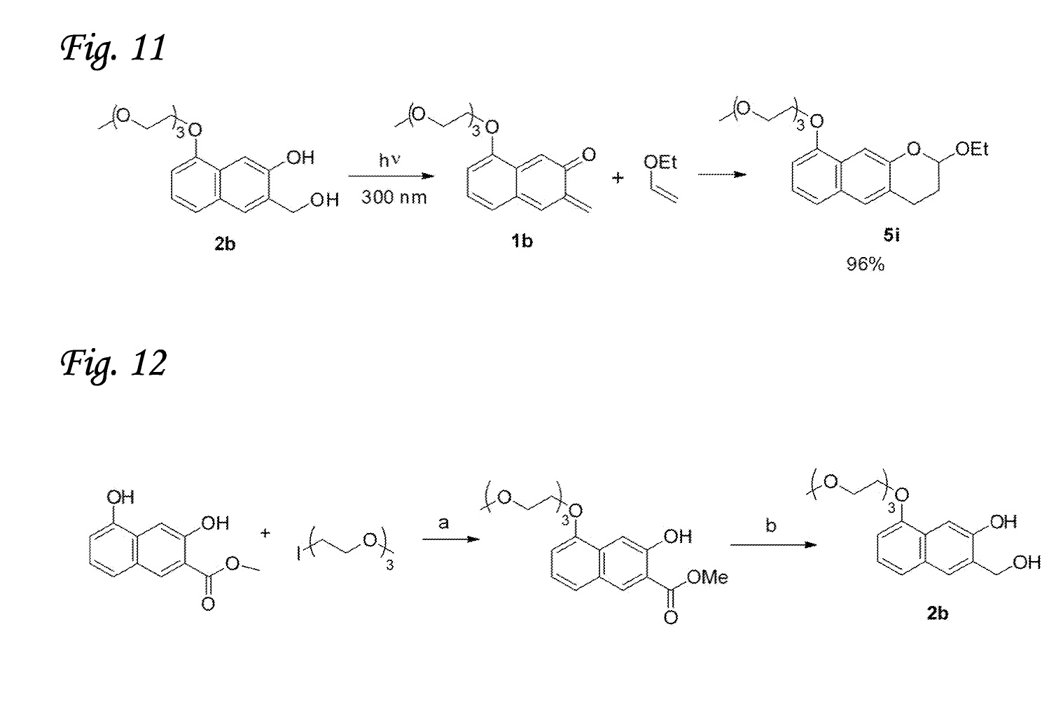

The second component for the ligation can be attached to the naphthalene ring of the precursor 2 via an appropriate linker (FIG. 1). For example, we have prepared 3-hydroxy-2-naphthalenemethanol modified at 8-position of a naphthalene ring with a tri-ethylene glycol linker (2b, FIG. 11). Upon irradiation of this precursor in the presence of ethyl vinyl ether (3a) in aqueous solution, the adduct 5h of the corresponding oNQM 1b to 3a is formed quantitatively (Table 5). This product was isolated in 96% yield and fully characterized.

TABLE 5
Chemical yields of ligation products upon photolysis of oNQM
precursors 2a-b in the presence of various vinyl ethers in 50% MeCN
in 0.01N aqueous phosphate buffer solution.^{a, b.}

| | | | Chemical yield of the |
|---|---|---|---|
| | | | ligation product |
| Olefin | oNQM Precursor | Ligation Product | (% conversion) |
| 3e | 2a | 5g | 94 ± 4 (>99) |
| 3f | 2a | 5h | 96 ± 2 (>99) |
| 3a | 2b | 5i | 96 ± 2 (>99) |

^{a}[Olefin] = 1.5 mM; [oNQM precursor] = 1 mM.
^{b}Irradiation wavelength = 300 nm.

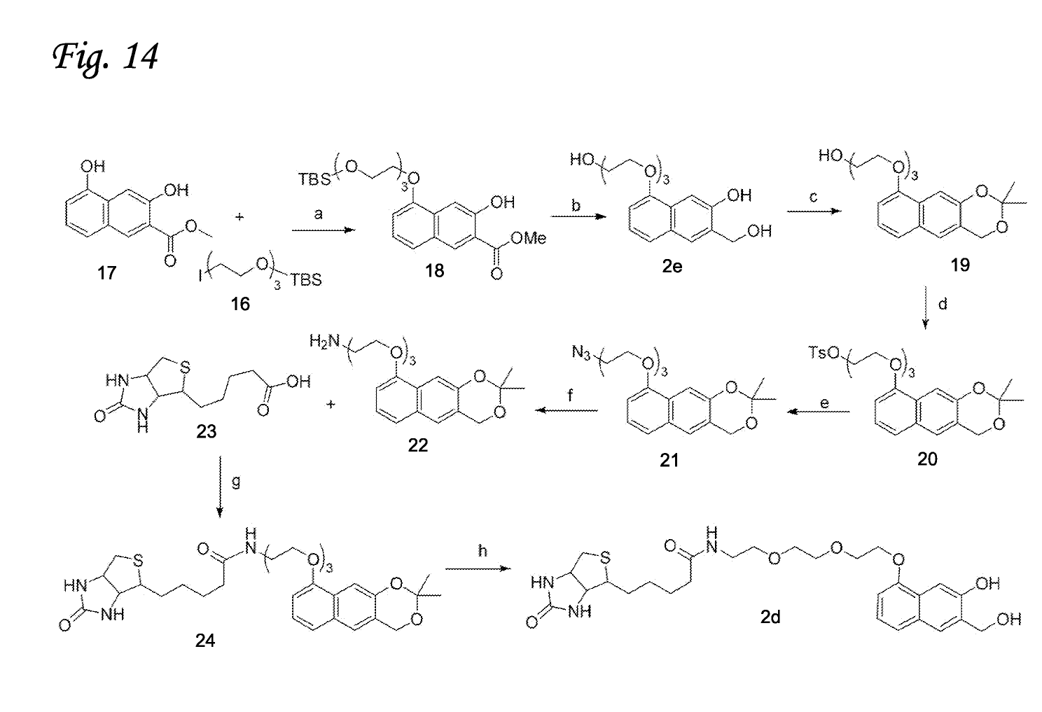

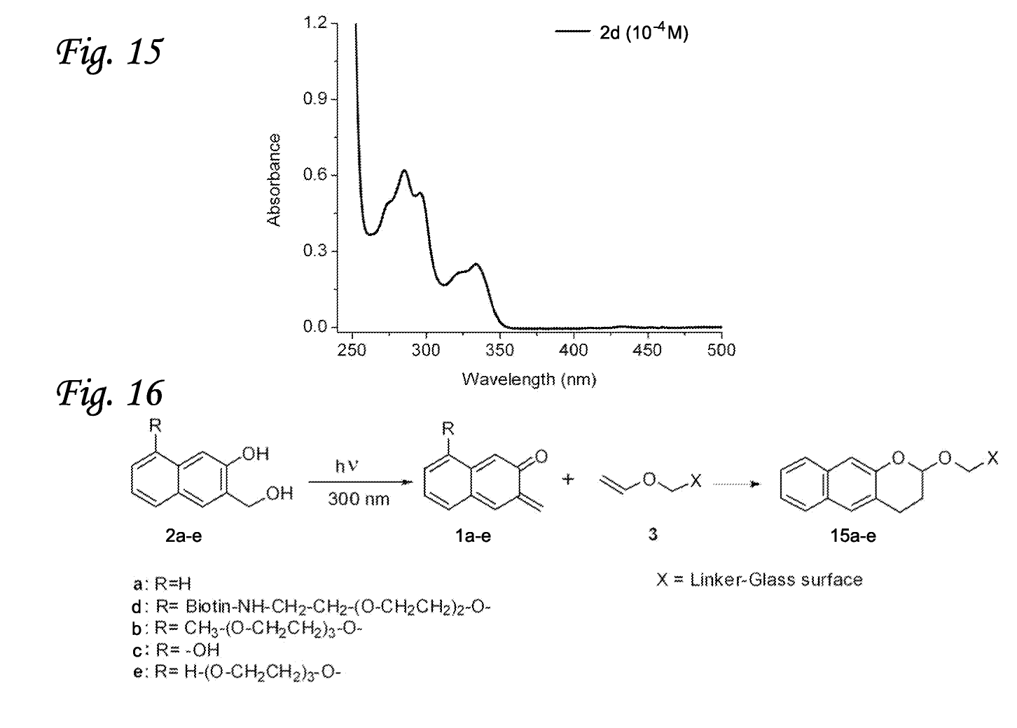

We have also shown that triethyleneglycol-derivatized 3-hydroxy-2-naphthalenemethanol can be further linked to biotin, producing the biotin-3-hydroxy-2-naphthalenemethanol conjugate 2d illustrated in FIG. 14. The UV Spectra of the biotin-3-hydroxy-2-naphthalenemethanol conjugate 2d is illustrated in FIG. 15.

In summary, we have demonstrated a very facile and selective photo-activatable click reaction that has high potential utility for light-triggered bioconjugation. The reaction is very selective towards electron rich polarized alkenes and demonstrates wide scope of reactivity that provides dual mode of ligation. The click tool compromising o-napthoquinone methides and vinyl ether system can be tailor made for a desired application depending on whether it requires ligation via labile C—O bond or permanent C—C bond. The selectivity of the click reaction can be useful in site specific labeling of biological system such as cell surface, proteins etc. Most importantly, the ligation can be achieved with very high spatial and temporal resolution due to the high rate of the reaction. The oNQM precursors and vinyl ethers are stable and do not react in the dark. High photostabilty of the hetero-Diels-Alder adduct completely allows for the elimination of secondary photochemistry after the click reaction. Currently in our lab, we have been investigating the utility of this click system as a technical tool for fluorescent labeling of biological systems and patterned immobilization of biomolecules on glass surface.

### Example 2

We envisaged that this photo-click reaction can be efficiently employed for the light-directed immobilization (patterning) of various substrates on the surface. For surface derivatization, one can adopt a conventional strategy that requires the oNQM precursor, 3-hydroxy-2-naphthalenemethanol derivative to be attached to the appropriate surface. Flood or patterned irradiation of such derivatized surface in the presence of a vinyl component carrying a payload substrate will result in immobilization of the latter. However, a more attractive from technological point of view immobilization/patterning method consists of the derivatization of the surface with vinyl ether moieties followed by the light directed attachment of substrates containing o-hydroxybenzyl alcohol or 3-hydroxy-2-naphthalenemethanol chromophore. (FIG. 16). Since the life-time of oNQM 1 in aqueous media is below 10 ms, diffusion of the photo-generated oNQM species from the site of irradiation is very limited. An example of the application of such technique is presented in this paper.

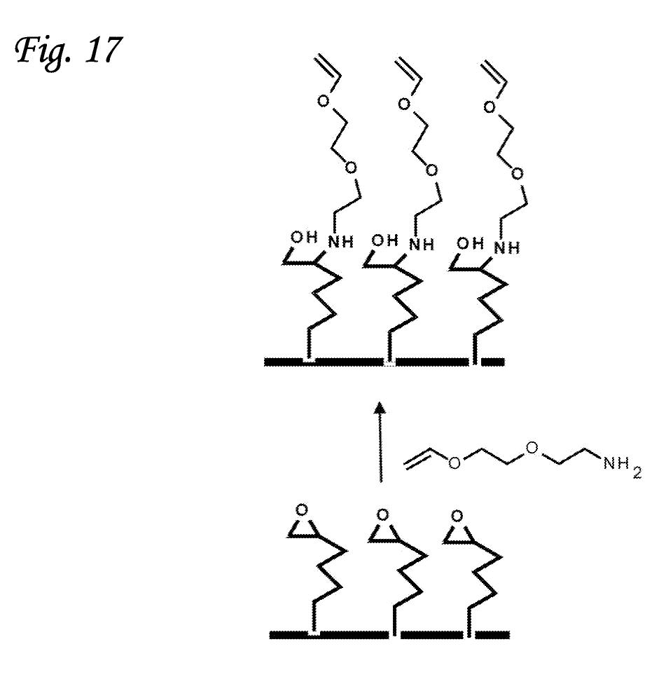

Commercial epoxide-functionalized glass slides were treated with 2-hydroxyethyl vinyl ether in the presence of catalytic amount of anhydrous p-toluenesulfonic acid or treated with 2-(2-(vinyloxy)ethoxy)ethanamine in dichloromethane at room temperature to yield vinyl ether functionalized glass surface. (FIG. 17). The resulting vinyl ether functionalized were covered with aqueous solution of containing biotin-3-hydroxy-2-naphthalenemethanol conjugate 2d (1×10⁻⁴ M), and irradiated with fluorescent UV lamps. The glass slides clicked biotin in this fashion were stained with fluorescently labeled FITC-Avidin and washed thoroughly by sonicating the glass slide in phosphate buffer solution followed by overnight incubation in the fresh phosphate buffer solution. The aforementioned extensive washing was required to clean up non-specific binding of FITC-Avidin on the glass surface.

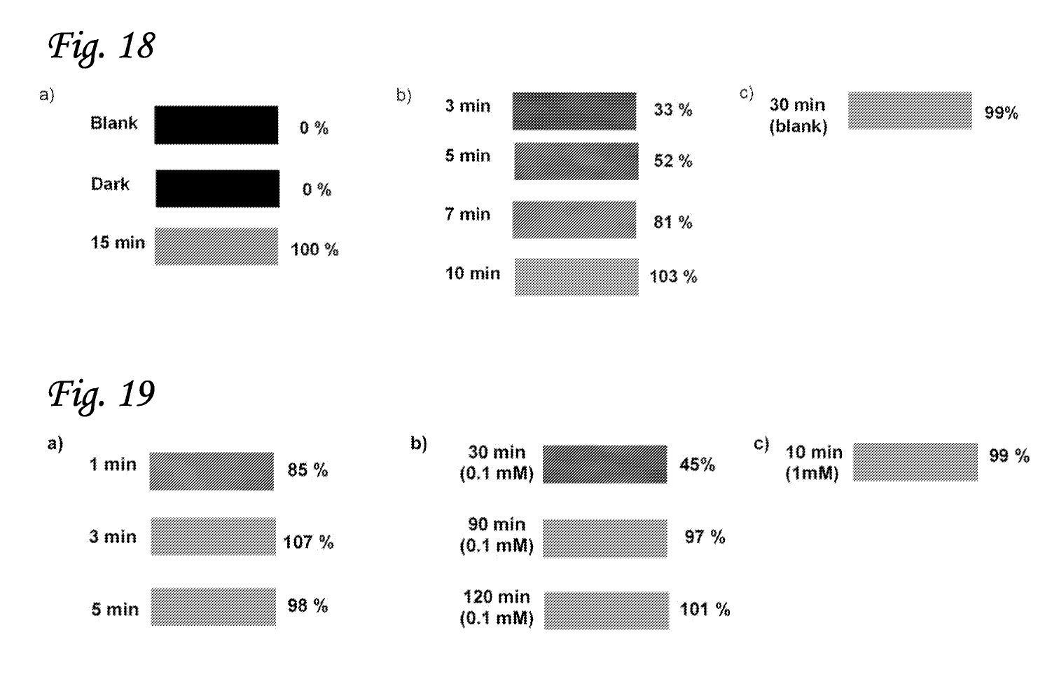

The degree of biotin functionalization as a function of irradiation time was monitored by following the fluorescence intensity of the resulting glass slides. The fluorescence intensity of the glass slide increases with the increase in irradiation time (using sixteen 8W 300 nm lamps) up to 10 minutes and the intensity saturates upon further irradiation. The high intensity of fluorescence recorded from these slides after 10 minutes photolysis indicates high density of functionalization. The saturation of fluorescence upon further irradiation (e.g. 15 minutes) can be attributed to the complete functionalization of vinyl ether group and the photostabilty of the resulting hetero-Diels-Alder adduct. The photostabilty of the hetero-Diels-Alder adduct is further confirmed by the fact that the fluorescence intensity remains the same even after 30 minutes irradiation of 10 minutes biotinylated sample in plain aqueous phosphate solution. (FIG. 18).

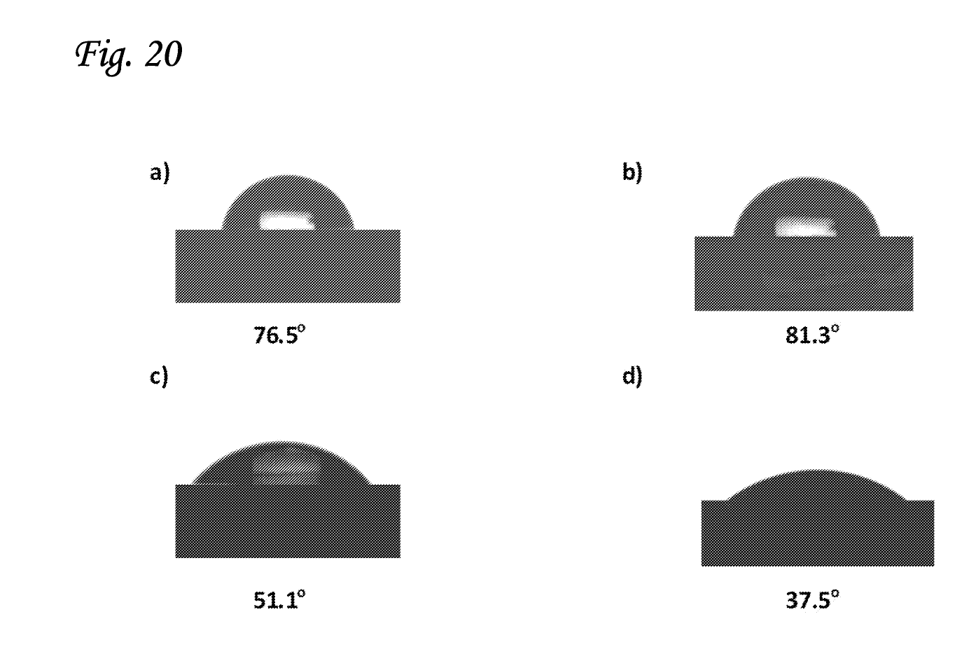

The irradiation time can be cut short in to 3 minutes by using medium pressure mercury lamp with 295 nm cut off glass filter. (FIG. 19). For practical applications, the utility of this reaction was also tested using 350 nm fluorescence lamps. Since the absorbance at 350 nm is low at experimental condition (0.1 mM, FIG. 15), either the substrate concentration should be increased to 1 mM or the irradiation should be increased to 90 minutes to get complete functionalization.

The surface derivatization of vinyl ether functionalized glass surface with various oNQM precursors was also studied as a function of its wetability. The water contact angle measurements were made with vinyl ether functionalized glass surface and the same after photo click reaction with oNQM precursor 2a, 2b, and 2d. FIG. 20 clearly demonstrates that the wettabilty change is minimal after derivatization with 2a and shows appreciable change in the case of 2b and 2d. This change in wettabilty after photo-click with 2d and 2b can be attributed to increased hydrophillicity of the surface due to the derivatization of glass surface with triethylene glycol appended biotin and triethylene glycol monomethyl ether respectively. Table 6 presents the comparison of water contact angles of commercially available epoxide slides (VWR # 66025-582) with the derivatized ones.

TABLE 6
Water contact angle measurements of glass slides with various
functional groups.

| Functional groups | Water contact angle |
|---|---|
| Commercially available epoxide (VWR) | 91.0 ± 2.5 |
| Vinyl ether + hydroxy group from opened epoxide | 77.0 ± 2.2 |
| 2a - vinyl ether adduct | 82.7 ± 1.9 |
| 2d - vinyl ether adduct | 38.0 ± 1.4 |
| 2b - vinyl ether adduct | 52.2 ± 1.5 |

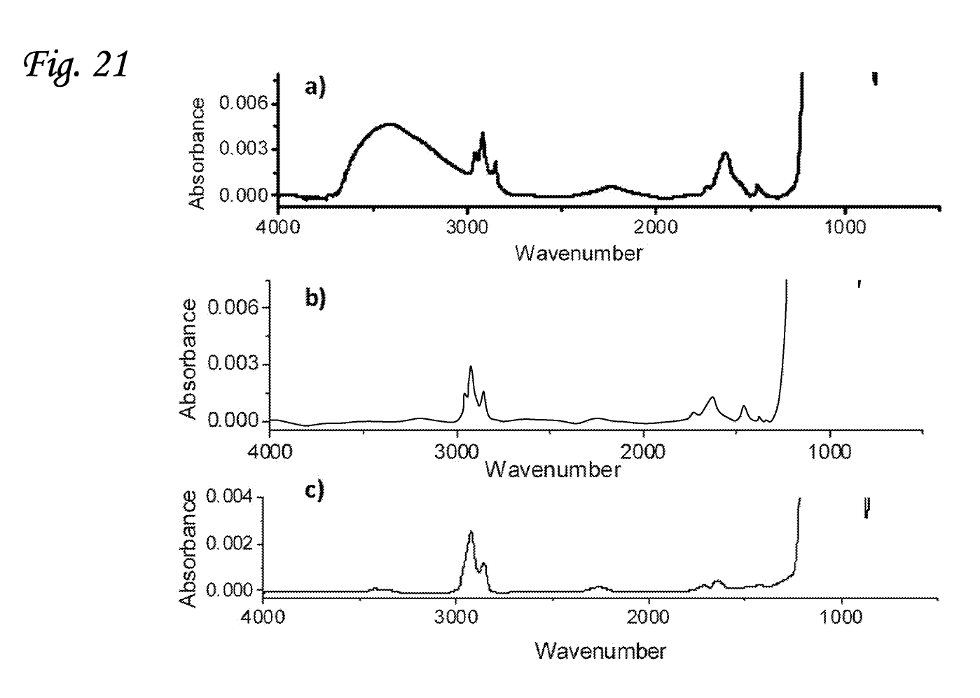

Attempts were also made to follow the surface derivatization with FTIR spectroscopy. (FIG. 21). The region below the wavenumber 1300 cm⁻¹ is completely masked by high intensity signals corresponds to silicon oxide bond. In the case of vinyl ether deravatized glass surface, only the signal corresponds to C—H stretching was clearly observed. The other peaks were too low to identify any other functional groups. However after photo-click reaction with oNQM precursor 2c, a strong broad signal centered at 3440 corresponds to non-hydrogen bonded phenolic OH was observed. As expected, the broad signal at 3440 was not observed after photo-click with 2a. These experiments stands as an additional supporting evidence for the efficient derivatization of glass surface using oNQM based photo click reaction.

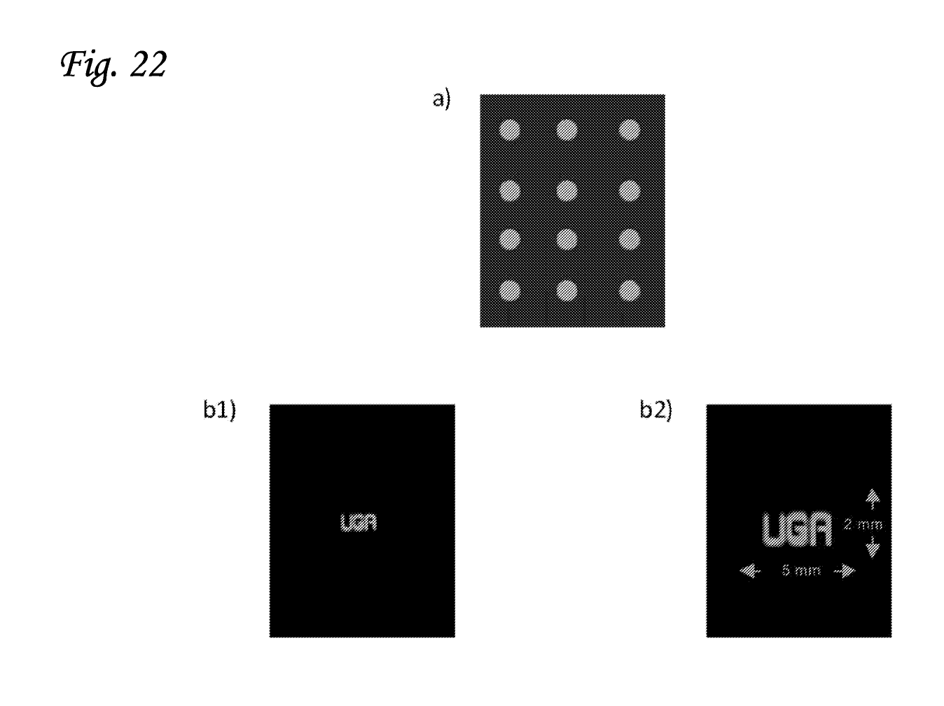

We have also tested two procedures for patterned functionalization of the slides. In the Method A, ca. 2 μL drops of the biotin-3-hydroxy-2-naphthalenemethanol conjugate 2d were placed on the vinyl ether-derivatized glass plate and irradiated with fluorescent UV lamp. The fluorescent image obtained after staining of the slide with fluorescently labeled FITC-Avidin, and washing is shown in FIG. 22a. In the Method B, the vinyl ether derivatized glass slide was placed into the solution, containing biotin-3-hydroxy-2-naphthalenemethanol conjugate 2d and irradiated through a “UGA” mask. The fluorescent image obtained after staining of the slide with fluorescently labeled FITC-Avidin, and washing is shown in FIG. 22b1.

As in the case of flood irradiated samples, the FITC-Avidin stained photo-clicked surfaces should be washed by sonicating the glass slides in phosphate buffer saline solution for 30 minutes followed by overnight incubation in fresh phosphate solution to avoid any non-specific binding of FITC-Avidin to glass surface. From technical point of view, a shorter washing procedure will enhance the efficacy of this photo-click system. This can be achieved by PEGlating the patterned biotin deravatized glass slides. For this purpose, the commercially available epoxide slide was treated with 2-(2-(vinyloxy)ethoxy)ethanamine (PEG 2 amine) to yield vinyl ether glass slide with PEG 2 functionality. This vinyl ether slide was taken for patterned surface derivatization following the procedure described in Method A. The patterned photo-clicked glass slides thus obtained was flood irradiated in an aqueous solution oNQM precursor 2e containing PEG3-OH functionality and then stained with labeled FITC-Avidin. The patterned surface deravatized glass slide with labeled FITC-Avidin was incubated in phosphate saline solution for 1 hour and then scanned in variable mode Typhoon imager.

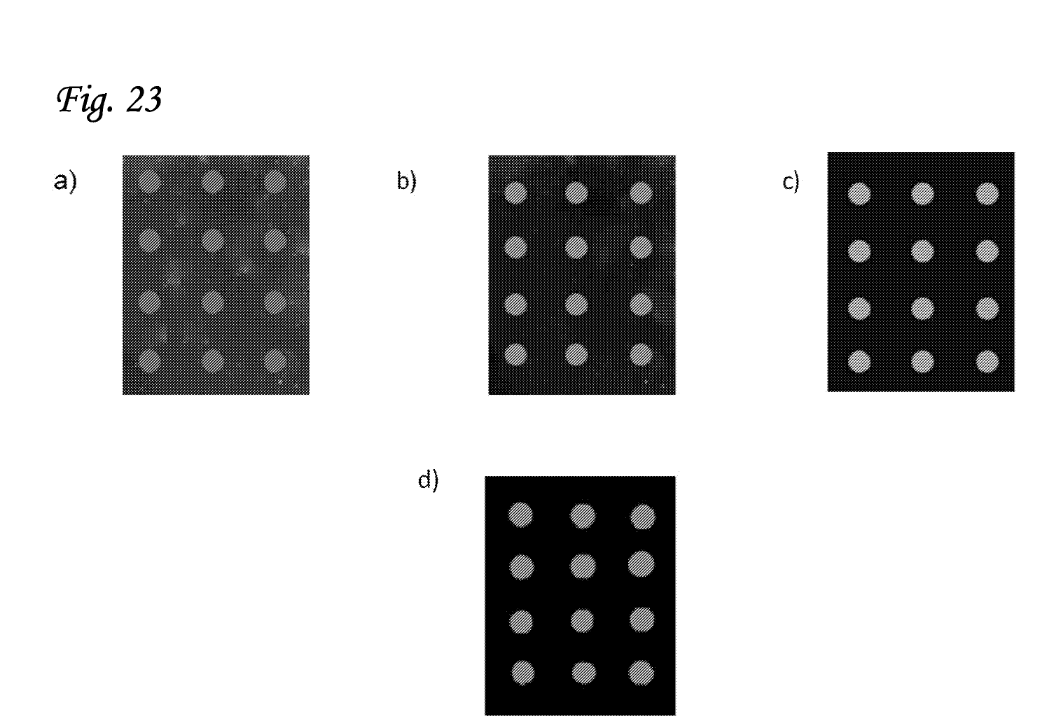

For comparative purposes, three more patterned biotin deravatized glass slides were prepared and stained with FITC-Avidin without PEGlation. (FIG. 23). The obtained FITC-Avidin labeled glass slides 1, 2 and 3 were incubated in phosphate buffer saline solution for 1 hour, 6 hours, and 16 hours respectively. FIG. 23d clearly demonstrates that PEGlation of patterned derivatized glass slides prevents non specific binding of FITC-Avidin and therefore a shorter washing procedure is enough for obtaining a clear pattern. Whereas longer washing duration (16 hours) was required to obtain similar pattern if the patterned derivatized surfaces are not PEGlated. FIGS. 23a and 23b demonstrates the non-specific binding of FITC-Avidin when non-PEGlated patterned derivatized surfaces are washed for shorter duration (1 hour and 6 hours.)

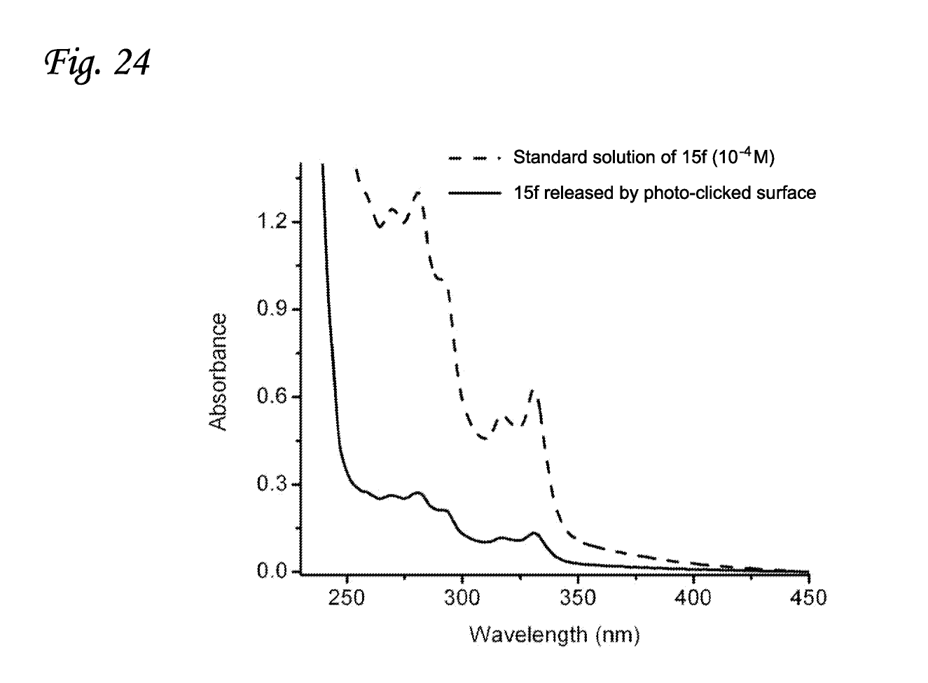

In all aforementioned experiments, the vinyl ether functionality was appended to the glass surface through the oxygen atom. As shown in FIG. 5, the hetero-Diels-Alder adduct formed after photo-click reaction is acid labile and can be hydrolyzed at pH 1. This permits complete detachment of the molecules that are previously immobilized on the glass surface upon dilute acid treatment and thereby allows to measure the concentration of fluorophore that can be immobilized by this photo-click system. For this purpose a commercially available epoxide glass slide of known molecular density (VWR # 16001-030, density=2×10¹³ molecules per mm², surface area=1875 mm²) was converted into vinyl ether functionalized glass slide and subsequently photo-clicked with oNQM 1a. The resulted photo-clicked surface that contains napthochroman 15a was then treated with 0.1N perchloric acid to trigger the release of 2-hydroxynaphthochroman 15f. The concentration of the released payload 15f was determined from its extinction coefficient at 281 nm by UV-Vis Spectroscopy (FIG. 24). It was calculated from three independent trials that about 70±4% of epoxide functionality were converted into payloads.

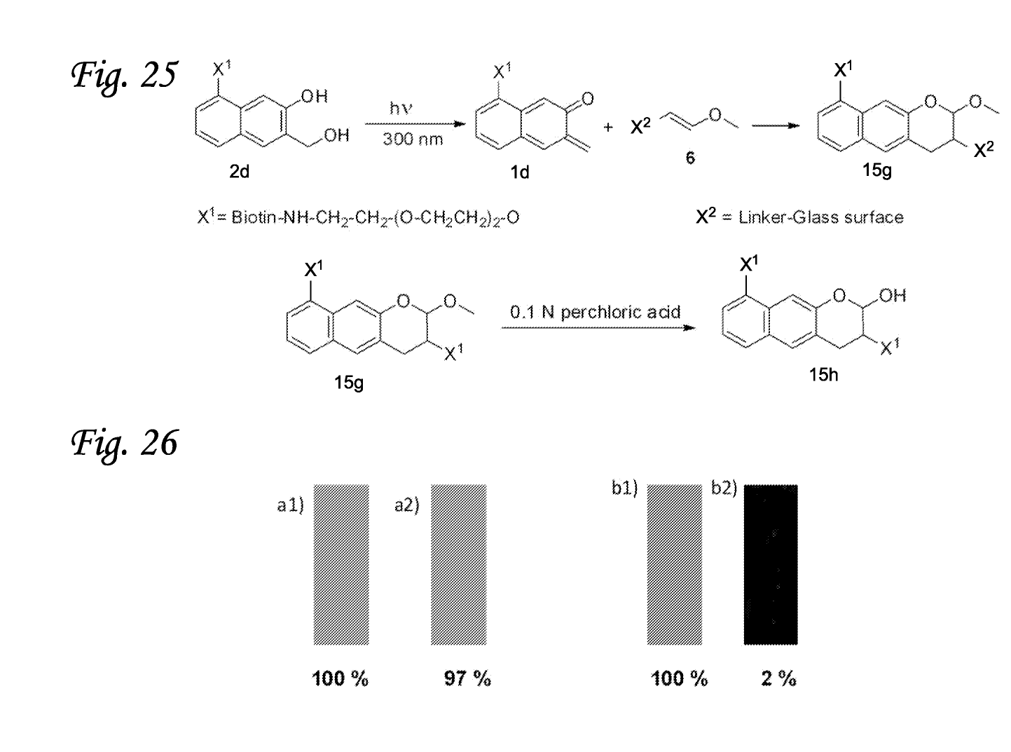

For the application that requires permanent ligation, the vinyl ether should be immobilized on the glass surface through its β-carbon (FIG. 25). This can be achieved by treating the commercially available epoxide glass slides with 6-methoxyhex-5-en-1-ol in the presence of catalytic amount of anhydrous p-toluenesulfonic acid. In this case, the payload remained immobilized on the glass surface even after the dilute acid hydrolysis. For illustrative purpose, two vinyl ether glass slides prepared from 6-methoxyhex-5-en-1-ol and were flood irradiated in an aqueous solution of 2d to yield biotinylated glass slides. One of the biotinylated glass slides was then incubated in 0.1N perchloric acid solution overnight. The hydrolyzed glass slides and unhydrolyzed glass slides were then stained with labeled FITC-Avidin, washed with phosphate buffer solution overnight and scanned in Typhoon imager. As presented in FIG. 26, the acid hydrolysis did not detach any biotin molecules and therefore the resulting fluorescence intensities of both the slides (a1 and a2) are very much the same.

Control experiments with the vinyl ether slides (immobilized via oxygen atom) prepared from 2-(2-(vinyloxy)ethoxy)ethanamine were performed. As clearly seen from the FIG. 26 (slide b1 and b2), the acid hydrolysis almost quantitatively detach all the biotin molecules and therefore the difference in fluorescence intensities between the hydrolyzed glass slide and unhydrolyzed glass slide is close to 100%

In summary, we have demonstrated a very facile and highly efficient photo-click reaction for light triggered surface derivatization. The reaction dynamics of the click reaction is very rapid to allow patterned surface derivatization despite the fact that the photoactive click pair is taken in solution. From the technical point of view, this is more advantageous than functionalize the glass surface with the photoactive component. Functionalization of glass surface with photoactive component involves complication such as filter effect, secondary photochemistry on the surface. Besides, taking photoactive component in the solution provides more control in tuning its absorption at any given wavelength. Hence one can tailor the irradiation time at any particular wavelength and thereby number of molecules need to be attached on the surface. Due to robustness of the click reaction, light can be used as on/off switch for the immobilization of the molecules. The wide scope of this click reaction permits us to immobilize the vinyl ether functionality either through the oxygen atom or β-carbon of the vinyl ether. Immobilization through oxygen atom allows us to detach the payload when needed. Whereas permanent ligation can be achieved by immobilizing the vinyl ether through the β-carbon.

### Example 3

General: All organic solvents were dried and freshly distilled before use. Flash chromatography was performed using 40-63 μm silica gel. Solutions for photochemical reaction were prepared using HPLC grade water and acetonitrile. Photoproducts were isolated from preparative scale reaction and were characterized by NMR, GC-MS and HRMS. The isolated pure photoproducts were then used as calibration standards for analytical scale reactions. Both preparative and analytical reactions were carried out using mini-Rayonet photochemical reactor equipped with 8 fluorescent UV lamps (4W, 254, 300, or 350 nm). Reaction mixtures after photolysis were analyzed by HPLC and chemical yields were determined from the calibration plot constructed using known standards of the pure product. Rate measurements were conducted using LKS.60 kinetic spectrometer (Applied Photophysics) equipped with Brilliant B Nd: YAG laser (pulse width=4 ns) fitted with 2^{nd} and 4^{th} harmonic generators. Substrate concentration for kinetics experiments was kept at ca 1×10 M for 2a and 0.04-0.05M for various olefins. Quantum efficiencies of photochemical reactions were measured by ferrioxalate actinometry. Buffer solutions for kinetic experiments were prepared using literature pKa values of the buffer acids and activity co-efficient recommended by Bates. (Bates, R. G. Determination of pH Theory and Practice; Wiley: New York, 1973; p 49). Quantum efficiencies of photochemical reactions were measured by ferrioxalate actinometry. (Murov et al., in: Handbook of Photochemistry, Marcel Dekker: New York, 1993, p. 299).

Materials: Ethyl vinyl ether, olefins 8-11, 2-hydroxyethyl vinyl ether (3b), isopropenyl methyl ether (3c) and triethyleneglycol monomethyl ether were purchased from VWR and used as received. Synthesis of ethyl(3-hydroxy-2-naphthalenyl)methyl ether (Arumugam et al., J. Am. Chem. Soc. 2009, 131, 11892), 3-hydroxy-2-naphthalenemethanol (2a) (Arumugam et al., J. Am. Chem. Soc. 2009, 131, 11892), phenyl vinyl ether 12, (Grobelny et al., J. Organomet. Chem. 2004, 689, 1580) enamine 13 (Klima et al., J. Org. Chem. 2007, 72, 6372) and 1-ethoxy-1-heptene (3d) (Ghribi et al. Tetrahedron Lett. 1984, 25, 3079) were previously reported in the literature.

Photolysis procedure: For the control experiments with olefins 3a and 8-12, solution of 2a (0.0029 mmol) and olefins 3a and 8-12 (0.29 mmol) in acetonitrile-water (1:1, 3 mL) was irradiated using mini-Rayonet photochemical reactor equipped with 16 fluorescent UV lamps (RPR-3000A°) for 3 minutes (15% conversion) and 20 minutes (>99% conversion). The same photolysis procedures were followed for the preparative and analytical scale ligation of oNQM precursor 2a and 2d with vinyl ethers 3a-f and enamine 13. The representative procedure for the ligation of 2a with vinyl with 3b is described below.

Preparative scale: Solution of 2a (0.3 mmol) and vinyl ether 3a (43 μL, 0.45 mmol) in acetonitrile-water (1:1, 300 mL) was irradiated using mini-Rayonet photochemical reactor equipped with 16 fluorescent UV lamps (RPR-2537A°) for 20 minutes. Photolysate was extracted with ethyl acetate, dried over sodium sulfate; and solvents were removed in vacuum.

DA adduct 5a: The adduct was separated by chromatography (20% EtOAc in hexane) to give 60 mg (87%) of 5a as colorless oil. ¹H NMR (400 MHz, CDCl₃): δ 7.86 (d, 7.6 Hz, 1H), 7.78 (d, 7.6 Hz, 1H), 7.66 (d, 8.8 Hz, 1H), 7.48-7.52 (m, 1H), 7.34-7.38 (m, 1H), 7.08 (d, 8.8 Hz, 1H), 5.38 (t, 2.8 Hz, 1H), 3.90-3.94 (m, 1H), 3.67-3.71 (m, 1H), 3.08-3.15 (m, 2H), 2.20-2.26 (m, 1H), 2.11-0.15 (m, 1H), 1.20 (t, 7. Hz, 3H); ¹³C NMR (100 MHz): 15.38, 17.69, 26.63, 64.02, 97.06, 114.45, 119.31, 122.22, 123.54, 126.47, 127.93, 128.60, 129.15, 133.70, 149.85; EI-MS m/z: 229 (M⁺+1), 228 (M+), 183, 182 (base peak), 181, 154, 153, 152, 128, 115, 102, 85, 72, 57, 55, 44; FW calc. (C₁₅H₁₆O₂): 228.1150, EI-HRMS found 228.1142.

DA adduct Sb: The adduct was separated by chromatography (10% EtOAc in hexane) to give 64 mg (87%) of 5b as colorless oil. ¹H NMR (400 MHz, CD₃CN): 7.70-7.78 (m, 2H), 7.62 (s, 1H), 7.37-7.41 (m, 1H), 7.31-7.34 (m, 1H), 7.24 (s, 1H), 5.42 (t, 2.8 Hz, 1H), 3.84-3.89 (m, 1H), 3.70-3.75 (m, 1H), 3.53-3.66 (m, 2H), 3.11 3.20 (m, 1H), 2.87-2.93 (m, 1H), 2.74 (t, J=6 Hz, 1H) 2.02-2.16 (m, 2H); ¹³C NMR (100 MHz, CD₃CN): 20.85, 26.65, 61.25, 70.11, 98.08, 111.66, 123.92, 125.37, 125.80, 126.44, 127.26, 127.87, 129.25, 133.76, 151.31; EI-MS m/z: 245 (10), 244 (80), 183 (40), 182 (70), 181 (100), 171 (10), 105 (10), 158 (70), 141 (75), 128 (80), 115 (20), 100 (20), 87 (75), 72 (25), 63 (15), 51 (10), 45 (30). FW calc. (C₁₅H₁₆O₃):244.1099, EI-HRMS: 244.1102.

DA adduct 5c: The adduct was separated by chromatography (10% EtOAc in hexane) to give 58 mg (85%) of 5c as colorless oil. ¹H NMR (400 MHz, CD₃CN): 7.69 (m, 2H), 7.55 (s, 1H), 7.69 (d, 8.3 Hz, 1H), 7.36 (m, 1H), 7.29 (m, 1H), 7.23 (s, 1H), 3.28 (m, 3H), 3.16-3.25 (m, 1H), 2.82-2.88 (m, 1H), 2.12-2.12 (m, 1H), 1.91-1.97 (m, 1H), 1.59 (t, 3H); ¹³C NMR (100 MHz, CD₃CN): 21.79, 21.75, 31.90, 48.66, 99.03, 111.68, 117.60, 123.87, 125.10, 125.71, 126.43, 127.23, 127.81, 129.26, 133.69, 151.82; EI-MS m/z: 229 (M⁺+1), 229(5), 228 (27), 213 (6), 197 (25), 196 (48), 195 (40), 181 (100), 168 (10), 157 (250, 150 (20), 141 (15), 128 (55), 115 (15), 102 (10), 90 (12), 76 (12), 63 (12), 51 (12), 43 (50). FW calc. (C₁₅H₁₆O₂): 228.1150, EI-HRMS: 228.1146.

DA adduct 5d: The adduct was separated by chromatography (10% EtOAc in hexane) to give 80 mg (88%) of 5d as colorless oil. ¹H NMR (400 MHz, CD₃CN): Diastereomer 1 (Major); 7.70-7.75 (m, 2H), 7.59 (s, 1H), 7.36-7.40 (m, 1H), 7.30-7.33 (m, 1H), 7.22 (s, 1H), 5.11 (d, J=3.2 Hz, 1H), 3.82-3.94 (m, 1H), 3.66-3.76 (m, 1H), 3.18-3.23 (m, 1H), 2.68-2.72 (m, 1H) 2.00-2.03 (m, 1H); 1.21-1.48 (m, 8H), 1.16 (t, J=6.8 Hz, 3H), 0.88 (t, J=6.8 Hz, 3H). Diastereomer 2 (Minor); 7.70-7.75 (m, 2H), 7.59 (s, 1H), 7.36-7.40 (m, 1H), 7.30-7.33 (m, 1H), 7.22 (s, 1H), 5.22 (d, J=3.2 Hz, 1H), 3.82-3.94 (m, 1H), 3.66-3.76 (m, 1H), 2.87-2.93 (m, 1H), 2.74-2.83 (m, 2H) 2.00-2.03 (m, 1H); 1.21-1.48 (m, 8H), 1.13 (t, J=6.8 Hz, 3H), 0.94 (t, J=6.8 Hz, 3H). ¹³C NMR (100 MHz, CD₃CN): Diastereomer 1 (Major); 13.62, 14.92, 22.59, 26.53, 26.65, 30.43, 32.96, 36.10, 63.97, 101.38, 111.39, 123.87, 124.68, 125.79, 126.49, 127.28, 128.36, 129.47, 133.88, 151.28 Diastereomer 2 (Minor); 13.62, 14.92, 22.59, 26.53, 26.65, 30.43, 32.96, 36.10, 63.97, 101.38, 111.39, 123.86, 125.53, 125.78, 126.44, 127.32, 127.90, 129.29, 133.87, 151.27EI-MS m/z: Diastereomer 1 (Major); 299 (20), 298 (100), 254 (10), 239 (80), 209 (10), 195 (40), 181 (75), 169 (15), 157 (30), 141 (20), 128 (35), 115 (10), 95 (15), 57 (10), 41 (17). Diastereomer 2 (Minor); 299 (20), 298 (100), 240 (15), 239 (75), 195 (30), 181 (70), 157 (25), 141 (20), 128 (35), 95 (10), 55 (10), 43 (12). FW calc. (C₂₀H₂₆O₂):298.1933, EI-HRMS:

DA adduct 5f: The adduct was separated by chromatography (10% EtOAc in hexane) to give 58 mg (85%) of 5f as colorless oil. ¹H NMR (400 MHz, CDCl₃): 7.67-7.71 (m, 2H), 7.54 (s, 1H), 7.35-7.38 (m, 1H), 7.29-7.34 (m, 1H), 7.23 (s, 1H), 5.17 (d. J=4 Hz, 1H), 3.06 3.10 (m, 1H), 2.95 (d, J=4 Hz, 1H), 2.61-2.65 (m, 1H), 1.14 (s, 1H) 1.03 (s, 3H); ¹³C NMR (100 MHz, CDCl₃): 23.98, 24.39, 32.84, 35.57, 98.97, 111.62, 123.79, 123.85, 125.72, 126.61, 127.21, 128.35, 129.42, 133.74, 149.98; EI-MS m/z: 229 (M⁺+1), 228(50), 195 (10), 183 (10), 158 (25), 157 (100), 128 (35), 115 (10), 71 (10), 43 (12). FW calc. (C₁₅H₁₆O₂): 228.1150, EI-HRMS: 228.1145

DA adduct 5g: The adduct was separated by chromatography (30% EtOAc in hexane) to give 136 mg (87%) of 5g as colorless oil. ¹H NMR (400 MHz, CD₃CN): 7.72 (d, 8 Hz, 1H), 7.69 (d, 8 Hz, 1H), 7.59 (m, 1H), 7.37 (t, 6 Hz, 1H), 7.29 (t, 6 Hz, 1H), 7.21 (s, 1H), 7.17 (d, 7.2 Hz, 2H), 6.73 (d, 7.2 Hz, 2H), 5.44 (t, 2.4 Hz, 1H), 4.26-4.32 (m, 1H), 4.03-4.11 (m, 3H), 3.95-4.00 (m, 1H) 3.05-3.12 (m, 1H), 2.96-3.00 (m, 1H), 2.78-2.90 (m, 1H), 2.01-2.11 (m, 2H), 1.34 (s, 9H); ¹³C NMR (100 MHz, CD₃CN): 20.67, 26.49, 27.66, 36.57, 51.82, 55.25, 66.78, 67.56, 97.85, 111.67, ELMS m/z: 521 (M+, 7), 466 (3), 447 (24), 421 (5), 404 (8), 362 (3), 333 (70), 227 (82), 183 (100), 155 (15), 153 (12), 128 (12), 107 (37), 91 (7), 78 (5). FW calc. (C₃₀H₃₅NO₇): 521.2414, EI-HRMS: 521.2408.

DA adduct 51h: The adduct was separated by chromatography (30% EtOAc in hexane) to give 100 mg (86%) of 5h as colorless oil. ¹H NMR (400 MHz, CD₃CN): 7.56 (s, 1H), 7.52 (s, 1H), 7.32 (d, J=8 Hz, 1H), 7.20 (t, J=8 Hz, 1H), 6.8 (d, J=7.2 Hz, 1H), 5.38 (t, 2.8 Hz, 1H), 4.25 (m, 2H), 3.83-3.92 (m, 3H), 3.65-3.71 (m, 3H), 3.57-3.63 (m, 4H), 3.45-3.48 (m, 2H), 3.29 (s, 3H) 3.05-3.15 (m, 1H), 2.82-2.92 (m, 1H), 2.05 (m, 2H), 1.16 (t, 7.2 Hz, 3H); ¹³C NMR (100 MHz, CD₃CN): 14.84, 20.87, 26.76, 58.17, 63.74, 64, 69.68, 70.35, 70.49, 70.81, 71.91, 97.58, 104.82, 106.36, 119.78, 123.89, 125.63, 125.86, 127.71, 129.15, 130.21, 151.17, 153.71; EI-MS m/z: 391 (15), 390 (50), 345 (20), 344 (100), 314 (5), 285 (10), 244 (20), 198 (35), 197 (32), 169 (15), 147 (17), 115 (13), 103 (15). FW calc. (C₂₂H₃₀O₆): 390.2042, EI-HRMS: 390.2048.

DA adduct 5i: The adduct was separated by chromatography (30% EtOAc in hexane) to give 150 mg (89%) of 51 as colorless oil. ¹H NMR (400 MHz, CDCl₃): Diastereomer 1 (major); 7.67-7.70 (m, 2H), 7.53 (s, 1H), 7.35 (t, J=7.6 Hz, 1H), 7.29 (t, J=7.6 Hz, 1H), 7.25 (d, J=4.8 Hz, 1H), 6.96 (d, J=8 Hz, 2H), 6.73 (d, J=8 Hz, 2H), 5.00-5.02 (m, 1H), 4.97 (d, J=2.4 Hz, 1H), 4.51-4.56 (m, 1H), 3.71 (s, 3H), 3.62-3.65 (t, J=6.4 Hz, 2H), 3.52 (s, 3H), 3.24-3.29 (m, 1H), 2.93-3.05 (m, 2H), 2.64-2.69 (m, 1H), 2.03-2.16 (m, 1H), 1.42 (s, 9H), 1.25-1.63 (m, 6H). Diastereomer 2 (Minor); 7.67-7.70 (m, 2H), 7.52 (s, 1H), 7.35 (t, J=7.6 Hz, 1H), 7.29 (t, J=7.6 Hz, 1H), 7.25 (d, J=4.8 Hz, 1H), 6.96 (d, J=8 Hz, 2H), 6.73 (d, J=8 Hz, 2H), 5.05 (d, J=2.4 Hz, 1H), 5.00-5.02 (m, 1H), 4.51-4.56 (m, 1H), 3.71 (s, 3H), 3.68-3.72, (t, J=6.4 Hz, 2H), 3.48 (s, 3H), 2.93-3.05 (m, 2H), 2.86-2.94 (m, 1H), 2.68-2.72 (m, 1H) 2.86-2.88 (m, 1H); 2.64-2.69 (m, 1H), 2.03-2.16 (m, 1H), 1.42 (s, 9H), 1.25-1.63 (m, 6H). ¹³C NMR (100 MHz, CDCl₃): Diastereomer 1 (major); 172.81, 155.47, 155.29, 150.52, 133.7, 130.61, 129.42, 128.47, 127.90, 127.20, 126.59, 125.70, 123.87, 123.77, 115.71, 111.85, 102.11, 80.34, 62.98, 55.95, 54.83, 52.46, 37.78, 35.89, 32.92, 30.35, 28.53, 26.59, 23.46. Diastereomer 2 (Minor); 172.81, 155.47, 155.29, 150.58, 133.7, 130.61, 129.26, 128.47, 127.83, 127.29, 126.54, 125.67, 124.74, 123.84, 115.71, 111.91, 100.83, 80.34, 63.04, 56.09, 54.83, 52.46, 37.78, 36.59, 33.03, 31.35, 28.53, 27.40, 23.29. FW calc. (C₃₃H₄₁NO₇): 563.2883, ESI-HRMS: 563.2898.

Analytical scale: Solution of 2a (0.003 mmol) and vinyl ether 3a (43 μL, 0.0045 mmol) in acetonitrile-water (1:1, 3 mL) was irradiated using mini-Rayonet photochemical reactor equipped with 16 fluorescent UV lamps (RPR-2537A°) for 20 minutes. An aliquot sample was drawn from the photolysate and analyzed in HPLC. Chemical yields were determined from the calibration plot constructed from the standard known isolated from preparative scale reactions.

Fluorescent measurements: Fluorescent spectra of 2a were recorded at λₑₓ=305 nm in doubly deionized water with the substrate concentration ca. 1×10⁻⁵ M using Varian steady state fluorimeter. The excitation source slits and the detector slits were set to 2 nm and 5 nm respectively. The fluorescence quantum yield were determined using 2-naphthol as the standard reference. (Wang et al., J. Org. Chem. 2002, 67, pp 2429; Berlman, I. B. Handbook of Fluorescence Spectra of Aromatic Molecules. Academic Press, New York, 1971, 473p). The fluorescent life time of the excited state of 2a were measured LKS.60 Applied Photophysics spectrometer using the fourth harmonic (266 nm) from the Brilliant B Nd:YAG laser as an excitation source with a pulse width of 7 ns.

Kinetic experiments: All kinetic rate measurements were made using LKS.60 Applied Photophysics spectrometer. Flash photolysis were initiated using the fourth harmonic (266 nm) from the Brilliant B Nd:YAG laser as an excitation source with a pulse width of 7 ns. Spectral changes were monitored at 310 nm using 100W xenon arc lamp, monochromotor and photomultiplier tube. Data were recorded on a digital oscilloscope and transferred to the computer for subsequent analysis. For a typical experiment samples of oNQM (1×10⁻⁴M) were prepared in a static quartz cell and purged with nitrogen for a period of 15 minutes prior to flash photolysis. The temperature of the sample solution during flash photolysis was maintained at 25±0.05° C. First order rate constant were obtained by least-square fitting of the observed exponential function for the QM formation and decay. Second order rate constant with NQM trapping reagents were determine from the plot of the concentration of the trapping reagents Vs the observed first order rate constants.

Synthesis of triethylene glycol-modified precursors 2b is outlined in FIG. 12. Preparation of triethylene glycol mono methyl ether and it's coupling to 3-hydroxy-2-naphthalenemethanol were achieved using literature procedures. All products were characterized by NMR, GC-MS and HRMS.

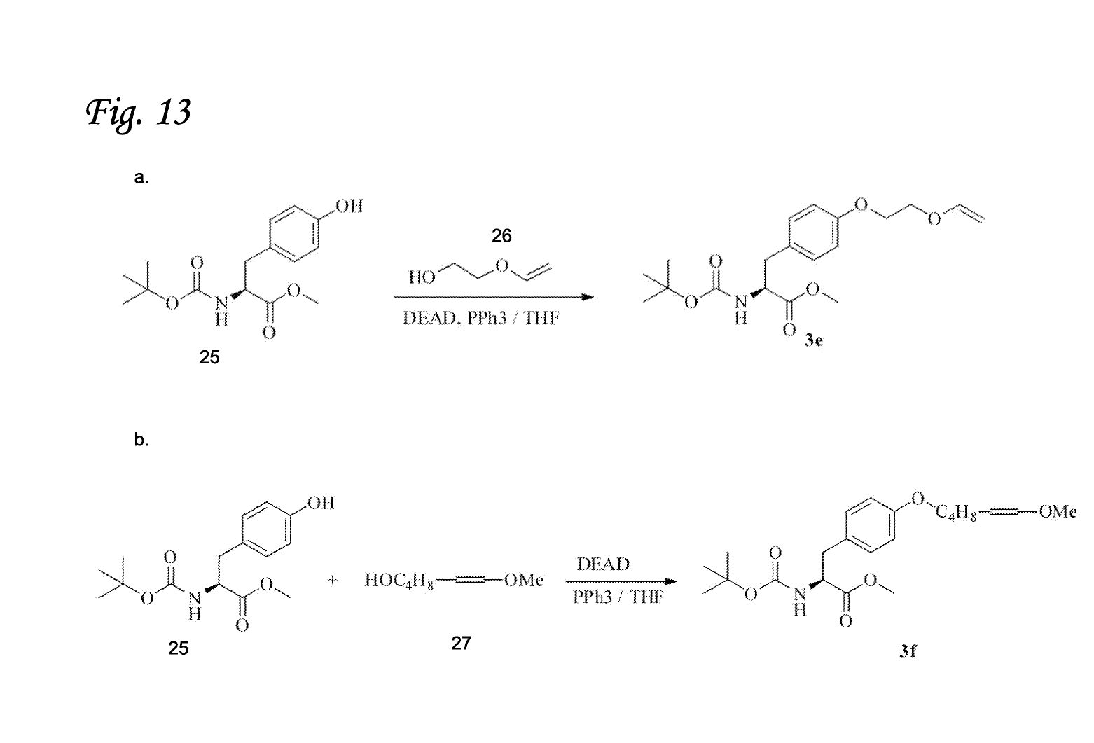

Synthesis of tyrosine appended vinyl ether 3e. The synthesis is illustrated schematically in FIG. 13a. About 1g (3.4 mmol) of 25 was taken in dry THF and to it 1.2 equivalent of PPh₃ (1.1.g, 4.1 mmol) were added at 0° C. To the reaction mixture, 1.2 equivalents of DEAD (710 mg, 4.1 mmol) was added and stirred for another 4 hours at room temperature. After the reaction is complete, it was quenched with water and the product was extracted in ethyl ether. The ether solution was washed with brine, dried over anhydrous magnesium sulfate and the solvent was removed under reduced pressure. The product was further purified column chromatography eluting with 5% ethyl acetate in chloroform to yield 1.1 g of 3e (87%). ¹H NMR (400 MHz, CDCl₃): 7.02 (d, J=4.8 Hz, 2H), 6.84 (d, J=4.8 Hz, 2H), 6.50-6.55 (m, 1H), 4.98-4.50 (m, 1H), 4.50-4.55 (m, 1H), 4.21-4.25 (m, 1H), 4.17-4.18 (m, 2H), 4.00-4.10 (m, 3H), 3.69 (s, 3H), 2.96-3.07 (m, 2H), 1.41 (s, 9H). ¹³C NMR (100 MHz, CDCl₃): 28.49, 37.71, 52.29, 54.78, 66.65, 80.04, 87.26, 114.98, 128.72, 130.46, 151.83, 155.29, 157.93, 172.56. EI-MS m/z: 365 (0.1), 309 (1), 292 (2), 248 (45), 232 (1), 206 (6), 177 (100), 162 (1), 151 (1), 135 (6), 117 (3), 107 (90), 88 (10), 73 (40), 71 (35). FW calc. (C₁₉H₂₇NO₆): 365.1838, EI-HRMS: 365.1827.

Synthesis of tyrosine appended vinyl ether 3e. The synthesis is illustrated schematically in FIG. 13b. About 750 mg (5.8 mmol) of 27 was taken in dry THF and to it 1.1 equivalent of 25 (1.88 g, 6.4 mmol), 1.2 equivalent of PPh₃ (1.81 g, 6.9 mmol) was added at 0° C. To the reaction mixture, 1.2 equivalents of DEAD (1.2 mg, 1.1 mL, 6.9 mmol) was added and stirred for another 4 hours at room temperature. After the reaction is complete, it was quenched with water and the product was extracted in ethyl ether. The ether solution was washed with brine, dried over anhydrous magnesium sulfate and the solvent was removed under reduced pressure. The product was further purified by column chromatography using 5% ether in dichloromethane to yield 1.65 g of the vinyl ether 3f (70%). ¹H NMR (400 MHz, CDCl₃) trans isomer: 7.02 (d, J=8.8 Hz, 2H), 6.82 (d, J=8.8 Hz, 2H), 6.32 (d, J=12.8 Hz, 1H), 4.96 (d, J=8 Hz, 1H), 4.71-4.77 (m, 1H), 4.52-4.57 (m, 1H), 3.93 (t, J=6 Hz, 2H), 3.72 (s, 3H), 3.51 (s, 3H), 2.97-3.08 (m, 2H), 1.98-2.03 (m, 2H), 2.75-1.82 (m, 2H), 1.48-1.55 (m, 2H) 1.43 (s, 9H); ¹³C NMR (100 MHz, CDCl₃) trans isomer: 27.37, 27.63, 28.53, 28.82, 37.68, 52.39, 54.76, 56.13, 68.00, 80.10, 102.82, 114.77, 127.95, 130.47, 147.54, 155.32, 158.41, 172.68. EI-MS m/z: 407 FW calc. (C₂₂H₃₃NO₆): 407.2308, EI-HRMS: 407.2298.

Synthesis of biotin-3-hydroxy-2-naphthalenemethanol conjugate 2d is outlined in FIG. 14.

Synthesis of 18: About 0.75 g (3.4 mmol) of 17 was taken in acetone (10 mL) and to it 1.2 equivalent of potassium carbonate (1.13 g, 8.17 mmol) was added. To the reaction mixture, 1.4 g (3.75 mmol) of 16 was added drop wise and refluxed for 4 hours. After the reaction is complete, the excess solvent was removed under reduced pressure and the residue was taken in ethyl ether. The ether solution was washed with brine, dried over anhydrous magnesium sulfate and the solvent was removed under reduced pressure. The product was further purified by column chromatography using 50% ethyl acetate in hexane to yield 1.03 g of 18 (65%). ¹H NMR (400 MHz, CDCl₃): 10.35 (s, 1H), 8.38 (s, 1H), 7.69 (s, 1H), 7.32 (d, 8 Hz, 1H), 7.15 (t, 7.6 Hz, 1H), 6.77 (d, 8 Hz 1H), 4.21-4.23 (m, 2H), 3.93-3.97 (m, 2H), 3.70-3.73 (m, 4H), 3.65-3.67 (m, 2H), 3.51-3.53 (m, 2H), 0.83 (s, 9H), 0.00 (s, 6H). ¹³C NMR (100 MHz, CDCl₃): −5.21, 18.41, 25.97, 52.58, 62.77, 68.06, 69.81, 70.89, 71.16, 72.78, 107.09, 107.31, 121.46, 123.80, 128.024, 130.61, 131.85, 153.33, 156.19, 170.36. EI-MS m/z: 464 (M+, 1), 449 (1), 417 (3), 409 (6), 407 (20), 376 (27), 375 (100), 331 (4), 287 (5), 259 (8), 257 (10), 243 (12), 213 (25), 185 (14), 169 (3), 157 (5), 129 (6), 101 (7), 88 (4), 73 (31). FW calc. (C₂₄H₃₆O₇Si): 464.2230, EI-HRMS: 464.2240.

Synthesis of 2e: About 1.2 equivalent of LiAlH₄ (0.100 g, 2.58 mmol) was taken in dry THF and to it 1g (2.15 mmol) of 18 was added at 0° C. The reaction mixture is warmed to room temperature and stirred for another 4 hours. After the reaction is complete, it was quenched with 5% HCl and the product was extracted in ethyl ether. The ether solution was washed with brine, dried over anhydrous MgSO₄ and the solvent was removed under reduced pressure. The crude mixture was taken in 10 mL of acetonitrile and stirred with 2.5 equivalents of aqueous HF. After complete deprotection of TBDMS group, the reaction mixture was taken in 100 mL of ethyl acetate and the organic extract was washed with saturated NaHCO₃, washed with brine, dried over anhydrous magnesium sulfate and the solvent was removed under reduced pressure. The product was further purified by column chromatography using 100% ethyl acetate to yield 0.59 g of 2e (85% yield). ¹H NMR (400 MHz, DMSO-d₈): 9.80 (s, 1H), 7.73 (s, 1H), 7.41 (s, 1H), 7.32 (d, 8 Hz, 1H), 7.12 (t, 7.6 Hz, 1H), 6.80 (d, 8 Hz 1H), 5.13 (t, J=5.6 Hz, 1H), 4.59-4.60 (m, 3H), 4.19-4.21 (m, 2H), 3.84-3.86 (m, 2H), 3.63-3.66 (m, 4H), 3.54-3.57 (m, 2H), 3.40-3.47 (m, 4H), ¹³C NMR (100 MHz, DMSO-d₈): 58.60, 60.32, 67.55, 69.23, 69.95, 70.22, 72.46, 102.79, 104.77. 119.89, 122.60, 125.17, 125.24, 128.72, 132.05, 152.75. EI-MS m/z: 322 (M⁺,90), 304 (25), 259 (5), 242 (5), 216 (12), 198 (25), 172 (7), 144 (50), 115 (70), 89 (100), 85 (60), 71 (5). FW calc. (C₁₇H₂₂O₆): 322.1416, EI-HRMS: 322.1413.

Synthesis of 19: About 0.5 g (1.55 mmol) of 2e was taken in acetone (15 mL) and to it 3 equivalents of 2,2-dimethoxypropane (0.480 g, 4.65 mmol) and catalytic amount of TsOH were added. The reaction mixture was stirred at room temperature overnight. The excess solvent was removed under reduced pressure and the residue was taken in dichloromethane. The dichloromethane solution was washed with brine, dried over anhydrous magnesium sulfate and the solvent was removed under reduced pressure. The product was further purified by column chromatography using 55% ethyl acetate in hexane to yield 0.465 g of 19 (90%). ¹H NMR (400 MHz, CDCl₃): 7.65 (s, 1H), 7.41 (s, 1H), 7.29 (d, 8 Hz, 1H), 7.18 (t, 7.6 Hz, 1H), 6.71 (d, 8 Hz 1H), 5.05 (s, 2H), 4.24-4.27 (m, 2H), 3.95-3.97 (m, 2H), 3.78-3.80 (m, 2H), 330-3.74 (m, 4H), 3.61-3.63 (m, 2H), 1.59 (s, 6H). ¹³C NMR (100 MHz, CDCl₃): 25.16, 61.38, 62.03, 68, 04470.51, 70.70, 71.24, 72.75, 100.03, 104.91, 107.27, 120.14, 121.74, 123.29, 123.88, 126.44, 129.72, 149.65, 153.83. DIP-EI-MS m/z: 362 (M+, 30), 304 (100), 273 (7), 259 (5), 216 (20), 198 (30), 174 (35), 172 (40), 144 (25), 115 (45), 85 (40), 87 (52), 73 (12). FW calc. (C₂₀H₂₆O₆): 362.1729, EI-HRMS: 362.1729.

Synthesis of 20: About 0.362 g (1 mmol) of 19 was taken in dichloromethane (7 mL) and to it 2 equivalents of triethylamine (0.2 g, 28 μL, 2 mmol) and 1.2 equivalents of p-tosyl chloride (228 mg, 1.2 mmol) were added. The reaction mixture was stirred at room temperature overnight. The excess solvent was removed under reduced pressure and the residue was taken in dichloromethane. The dichloromethane solution was washed with brine, dried over anhydrous MgSO₄ and the solvent was removed under reduced pressure. The product was further purified by column chromatography using 75% ethyl acetate in hexane to yield 0.423 mg of 20 (82%). ¹H NMR (400 MHz, CD₃CN): 7.77 (d, 8 Hz, 2H), 7.53 (s, 1H), 7.49 (s, 1H), 7.34-7.39 (m, 3H), 7.24 (t, 7.6 Hz, 1H), 6.82 (d, 8 Hz 1H), 5.05 (s, 2H), 4.21-4.24 (m, 2H), 4.11-4.12 (m, 2H), 3.88-3.90 (m, 2H), 3.63-3.64 (m, 4H), 3.53-3.55 (m, 2H), 2.39 (s, 3H), 1.55 (s, 6H). ¹³C NMR (100 MHz, CD₃CN): 20.95, 24.64, 61.07, 68.35, 68.62, 69.76, 70.30, 70.59, 70.75, 100.24, 105.37, 106.45, 120.13, 122.39, 123.85, 124.21, 126.32, 128.07, 129.87, 130.31, 133.33, 145.63, 149.99, 153.82. EI-MS m/z: 515 (M⁺, 3), 458 (15), 424 (12), 422 (12), 368 (40), 366 (42), 323 (7), 287 (10), 259 (50), 242 (32), 199 (50), 198 (40), 171 (25), 144 (25), 115 (60), 109 (90), 107 (100), 91 (25). FW calc. (C₂₇H₃₂O₈S): 516.1818, EI-HRMS: 516.1812.

Synthesis of 21: About 0.4 g (0.78 mmol) of 20 was taken in N,N-dimethylformamide (5 mL) and to it 2 equivalents of sodium azide (0.06 g, 0.94 mmol) was added. The reaction mixture was stirred at 60° C. overnight. The excess solvent was removed under reduced pressure and the residue was taken in ether. The ether solution was washed with brine, dried over anhydrous magnesium sulfate and the solvent was removed under reduced pressure. The product was further purified by column chromatography using 40% ethyl acetate in hexane to yield 0.240 mg of 21 (80%). ¹H NMR (400 MHz, CD₃CN): 7.58 (m, 2H), 7.35 (d, 8 Hz, 1H), 7.24 (t, 7.6 Hz, 1H), 6.83 (d, 8 Hz 1H), 5.06 (s, 2H), 4.24-4.26 (m, 2H), 3.93-3.95 (m, 2H), 3.73-3.76 (m, 2H), 3.66-3.67 (m, 4H), 3.66-3.69 (m, 2H), 1.57 (s, 6H). ¹³C NMR (100 MHz, CD₃CN): 24.64, 50.93, 61.09, 68.36, 69.78, 69.94, 70.58, 70.90, 100.24, 105.36, 106.47, 120.13, 122.41, 123.86, 124.22128.01, 129.88, 150.02, 153.85. EI-MS m/z: 387 (M⁺, 30), 359 (40), 318 (7), 301 (100), 260 (15), 212 (50), 199 (50), 185 (35), 170 (35), 144 (38), 129 (36), 115 (77), 73 (92). FW calc. (C₂₀H₂₅N₃O₅): 387.1794, EI-HRMS: 387.1787.

Synthesis of 22: About 2 equivalent of LiAlH₄ (0.038 g, 1 mmol) was taken in dry ethyl ether and to it 0.2 g (0.52 mmol) of 21 was added at 0° C. The reaction mixture is warmed to room temperature and stirred for another 4 hours. After the reaction is complete, it was quenched with 5% HCl and the product was extracted in ethyl ether. The ether solution was washed with brine, dried over anhydrous magnesium sulfate and the solvent was removed under reduced pressure to yield 152 mg (81%) of the amine 22 and was taken to next step without any purification. ¹H NMR (400 MHz, CD₃CN): 7.51 (s, 1H), 7.49 (m, 2H), 7.33 (d, 8 Hz, 1H), 7.23 (t, 7.6 Hz, 1H), 6.80 (d, 8 Hz 1H), 5.03 (s, 2H), 4.20-4.22 (m, 2H), 3.88-3.90 (m, 2H), 3.68-3.70 (m, 2H), 3.59-3.60 (m, 2H), 3.41-3.43 (m, 2H), 2.73 (br s, 2H), 2.10 (br s, 2H), 1.55 (s, 6H). ¹³C NMR (100 MHz, CD₃CN): 26.08, 41.61, 62.49, 69.76, 71.75, 72.21, 72.27, 101.66, 106.77, 107.90, 121.57, 123.79, 125.27, 125.64, 127.74, 131.28, 151.41, 156.24. EI-MS m/z: 361 (M⁺, 100), 343 (7), 326 (5), 303 (75), 286 (18), 260 (7), 244 (12), 228 (15), 201 (37), 186 (60), 172 (37), 144 (26), 128 (26), 115 (70), 103 (6), 88 (12), 70 (35). FW calc. (C₂₀H₂₇NO₅): 361.1889, EI-HRMS: 361.1889.

Synthesis of 24: About 56 mg of d-biotin (0.23 mmol) was taken in 5 mL dry N,N-dimethyl formamide and to it added 1.2 equivalents of EDC.HCl and catalytic amount of DMAP were added. To this solution, 90 mg (0.25 mmol) of the amine 22 in 2 mL of N,N-dimethyl formamide was added drop wise and the mixture was stirred for 12 hours at room temperature. After the reaction is complete, N,N-dimethyl formamide was removed in vacuum. The residue is taken dichloromethane, washed with NaHCO₃ solution, brine dried over anhydrous magnesium sulfate and the solvent was removed under reduced pressure. The product was further purified by column chromatography using 10% methanol in dichloromethane to yield 0.12 mg of 24 (83%). ¹H NMR (400 MHz, CD₃CN): 7.62 (s, 1H), 7.42 (s, 1H), 7.28 (t, 8 Hz, 1H), 7.19 (t, 8 Hz, 1H), 6.92 (t, 8 Hz, 1H), 6.77 (s, 1H), 6.72 (d, 8 Hz 1H), 5.87 (s, 1H), 5.04 (s, 2H), 4.36-4.39 (m, 1H), 4.24-4.25 (m, 2H), 4.08-4.11 (m, 1H), 3.93-3.94 (m, 2H), 3.65-3.67 (m, 2H), 3.56-3.59 (m, 2H), 3.41-3.43 (m, 2H), 2.91-2.93 (m, 1H), 2.74-2.78 (m, 1H), 2.63-2.66 (m, 2H), 2.15 (t, 7.8 Hz, 2H), 1.55 (s, 6H), 1.53-1.63 (m, 4H), 1.27-1.30 (m, 2H). ¹³C NMR (100 MHz, CD₃CN): 25.21, 25.75, 28.26, 36.1839.40, 40.70, 60.39, 61.38, 61.91, 68.11, 70.03, 70.24, 70.43, 70.93, 100.10, 104.98, 107.14, 120.27, 121.85, 123.42, 123.97, 126.36, 129.72, 149.72, 153.76, 164.35, 173.53. FW calc. (C₃₀H₄₁N₃O₇S): 587.2665, EI-HRMS: 587.2676.

Synthesis of 2d: About 100 mg (0.17 mmol) of 24 was taken 5 mL of methanol and to it 60 mg of Amberlyst was added and stirred for 2 hours at room temperature. The amberlyst resin was filtered through cotton plug and the methanol solution was passed through short silica gel to yield 93 mg analytically pure biotinylated 3-hydroxymethylnaphthalene-2-ol chromophore (2d) in 95% yield. ¹H NMR (400 MHz, d-4-Methanol): 7.90 (t, 8 Hz, 1H), 7.73 (s, 1H), 7.52 (s, 1H), 7.32 (d, 8 Hz, 1H), 7.14 (t, 8 Hz, 1H), 6.75 (d, 8 Hz 1H), 4.87 (s, 2H), 4.30-4.33 (m, 1H), 4.20-4.22 (m, 2H), 4.09-4.07 (m, 1H), 3.92-3.93 (m, 1H), 3.73-3.74 (m, 2H), 3.62-3.64 (m, 2H), 3.52-3.55 (m, 2H), 3.32-3.36 (m, 2H), 2.96-2.99 (m, 1H), 2.74-2.79 (m, 1H), 2.60-2.63 (m, 2H), 2.12 (t, 7.8 Hz, 2H), 1.42-1.62 (m, 4H), 1.23-1.32 (m, 2H). ¹³C NMR (100 MHz, d-4-Methanol): 25.60, 28.23, 28.49, 35.58, 39.23, 39.88, 55.72, 60.21, 62.10, 67.89, 69.48, 69.89, 70.14, 70.82, 96.13, 103.55, 105.06, 120.30, 122.84, 126.27, 126.43, 129.68, 130.74, 153.23, 153.40, 164.82, 174.97. FW calc. (C₂₇H₃₇N₃O₇S): 547.2352, EI-HRMS: 547.2349.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claim

What is claimed is:

1. A method for labeling a substrate, the method comprising:
generating an o-quinone methide attached to a surface of a substrate and having the formula: 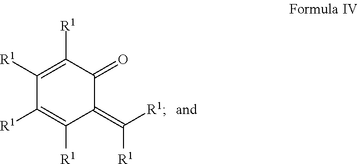
contacting the o-quinone methide with a polarized olefin having the formula: 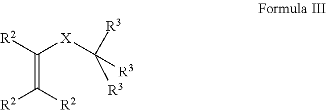
under conditions effective to form a hetero-Diels-Alder adduct, wherein:
each R¹ is independently H, halogen, or an organic group, with the proviso that at least one R¹ comprises a linker group attached to the surface of the substrate;
each R² is independently H or an organic group;
each R³ is independently H, halogen, or an organic group;
X is O or NR⁴, wherein R⁴ is H or an organic group;
optionally, two or more R¹ groups may be combined to form one or more rings;
optionally, two or more R² groups may be combined to form one or more rings;
optionally, two or more R³ and/or R⁴ groups may be combined to form one or more rings; and
with the proviso that at least one R², R³, or R⁴ comprises a detectable label.

2. The method of claim 1 wherein the o-quinone methide is an o-naphthoquinone methide having one of the formulas:

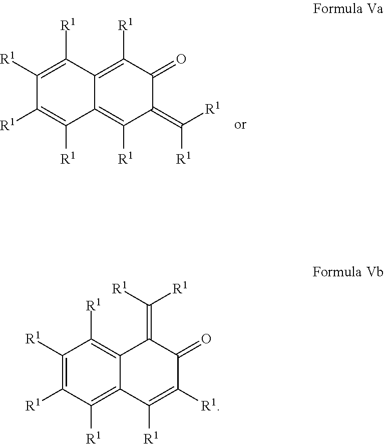

3. The method of claim 1 wherein the substrate comprises a planar surface or a bead.

4. The method of claim 1 wherein the substrate is selected from the group consisting of glass, quartz, silica, a metal, a semi-conductor, a polymer, a membrane, a liposome, a micelle, a macromolecule, a biomaterial, and combinations thereof.

5. The method of claim 4 wherein the biomaterial is selected from the group consisting of a virus, a small multicellular organism, DNA, RNA, a peptide, a polypeptide, a protein, a carbohydrate, a lipid, tissue, and combinations thereof.

6. The method of claim 1 wherein the label is detectable by method selected from the group consisting of fluorescence, phosphorescence, radiation detection, optical methods, electrochemical methods, surface plasmon resonance imaging (SPRi), and combinations thereof.

7. The method of claim 1 wherein the detectable label comprises a probe.

8. The method of claim 7 wherein the probe comprises DNA, a peptide, a polypeptide, a protein, or a combination thereof.

9. The method of claim 1 wherein the conditions effective to form the hetero-Diels-Alder adduct comprise contacting in an aqueous solution, suspension, or dispersion.

10. The method of claim 1 wherein R³ or R⁴ comprises the detectable label, and the method further comprises removing the detectable label from the surface of the substrate by hydrolyzing the C—X bond.

11. A method for labeling a substrate, the method comprising:
providing a precursor compound attached to a surface of a substrate and having the formula: 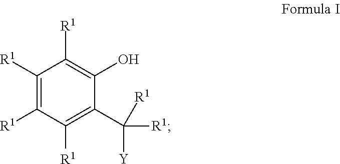
irradiating the precursor compound under conditions effective to form an o-quinone methide having the formula: 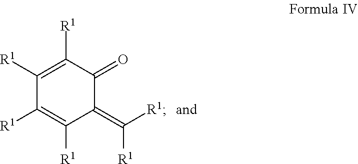
contacting the o-quinone methide with a compound having the formula 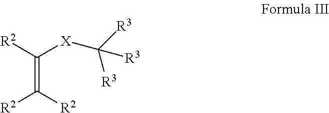
under conditions effective to form a hetero-Diels-Alder adduct, wherein:
each R¹ is independently H, halogen, or an organic group, with the proviso that at least one R¹ comprises a linker group attached to the surface of the substrate;
each R² is independently H or an organic group;
each R³ is independently H, halogen, or an organic group;
X is O or NR⁴;
Y is OR⁵, NR⁵₂, NR⁵₃⁺ (Z_{1/q})⁻ wherein Z is an anion having a negative charge of q;
each R⁵ is independently H or an organic group,
optionally, two or more R¹ groups may be combined to form one or more rings;
optionally, two or more R² groups may be combined to form one or more rings;
optionally, two or more R³ and/or R⁴ groups may be combined to form one or more rings;
optionally, two or more R⁵ groups may be combined to form one or more rings; and
with the proviso that at least one R², R³, or R⁴ comprises a detectable label.

12. The method of claim 11 wherein the precursor compound has one of the formulas: 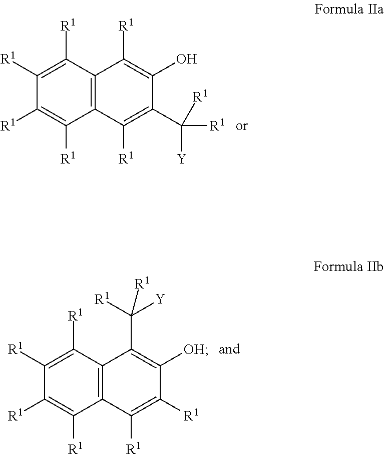
wherein irradiating the precursor compound under conditions effective to form an o-quinone methide forms an o-naphthoquinone methide having one of the formulas:

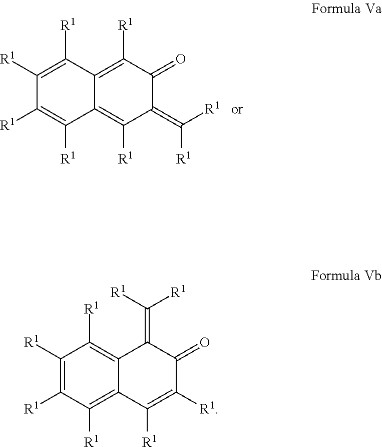

13. The method of claim 11 wherein the precursor compound is irradiated in the presence of the polarized olefin.

14. The method of claim 11 wherein irradiating the precursor compound comprises pattern-wise irradiating the substrate to provide a pattern-wise labeled surface of the substrate.

15. A method for labeling a substrate, the method comprising:
generating an o-quinone methide having the formula: 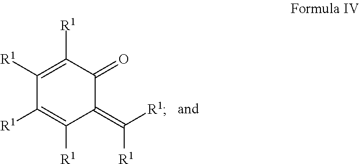
contacting the o-quinone methide with a polarized olefin attached to a surface of a substrate and having the formula: 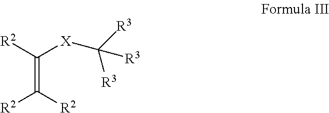
under conditions effective to form a hetero-Diels-Alder adduct, wherein:
each R¹ is independently H, halogen, or an organic group, with the proviso that at least one R¹ comprises a detectable label;
each R² is independently H or an organic group;
each R³ is independently H, halogen, or an organic group;
X is O or NR⁴, wherein R⁴ is H or an organic group;
optionally, two or more R¹ groups may be combined to form one or more rings;
optionally, two or more R² groups may be combined to form one or more rings;
optionally, two or more R³ and/or R⁴ groups may be combined to form one or more rings; and
with the proviso that at least one R², R³, or R⁴ comprises a linker group attached to the surface of the substrate.

16. The method of claim 15 wherein the o-quinone methide is an o-naphthoquinone methide having one of the formulas:

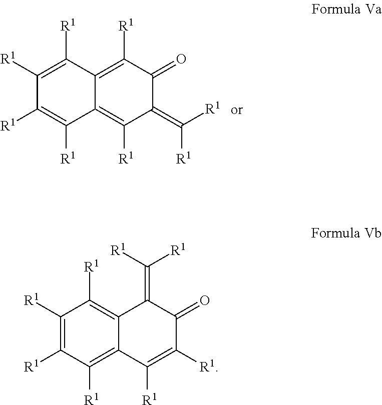

17. The method of claim 15 wherein the substrate comprises a planar surface or a bead.

18. The method of claim 15 wherein the substrate is selected from the group consisting of glass, quartz, silica, a metal, a semi-conductor, a polymer, a membrane, a liposome, a micelle, a macromolecule, a biomaterial, and combinations thereof.

19. The method of claim 18 wherein the biomaterial is selected from the group consisting of a virus, a small multicellular organism, DNA, RNA, a peptide, a polypeptide, a protein, a carbohydrate, a lipid, tissue, and combinations thereof.

20. The method of claim 15 wherein the label is detectable by a method selected from the group consisting of fluorescence, phosphorescence, radiation detection, optical and electrochemical methods, surface plasmon resonance imaging (SPR¹), and combinations thereof.

21. The method of claim 15 wherein the detectable label comprises a probe.

22. The method of claim 21 wherein the probe comprises DNA, a peptide, a polypeptide, a protein, or a combination thereof.

23. The method of claim 15 wherein the conditions effective to form the hetero-Diels-Alder adduct comprise contacting in an aqueous solution, suspension, or dispersion.

24. The method of claim 15 further comprising removing the detectable label from the surface of the substrate by hydrolyzing the C—X bond.

25. A method for labeling a substrate, the method comprising:
providing a precursor compound having the formula: 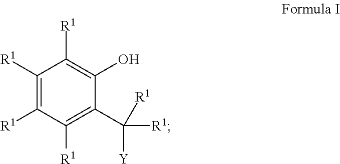
irradiating the precursor compound under conditions effective to fowl an o-quinone methide having the formula: 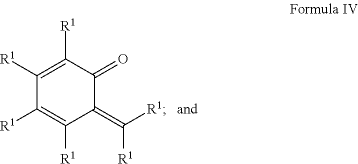
contacting the o-quinone methide with a polarized olefin attached to a surface of a substrate and having the formula: 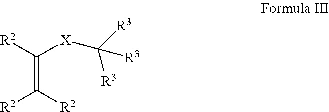
under conditions effective to form a hetero-Diels-Alder adduct, wherein:
each R¹ is independently H, halogen, or an organic group, with the proviso that at least one R¹ comprises a detectable label;
each R² is independently H or an organic group;
each R³ is independently H, halogen, or an organic group;
X is O or NR⁴;
Y is OR⁵, NR⁵₂, NR⁵₃⁺ (Z_{1/q})⁻ wherein Z is an anion having a negative charge of q;
each R⁵ is independently H or an organic group;
optionally, two or more R¹ groups may be combined to form one or more rings;
optionally, two or more R² groups may be combined to form one or more rings;
optionally, two or more R³ and/or R⁴ groups may be combined to form one or more rings;
optionally, two or more R⁵ groups may be combined to form one or more rings; and
with the proviso that at least one R², R³, or R⁴ comprises a linker group attached to the surface of the substrate.

26. The method of claim 25 wherein the precursor compound has one of the formulas: 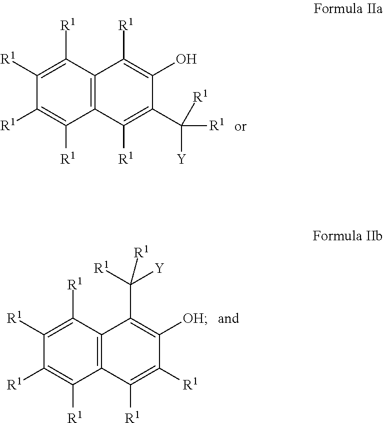
wherein irradiating the precursor compound under conditions effective to form an o-quinone methide forms an o-naphthoquinone methide having one of the formulas:

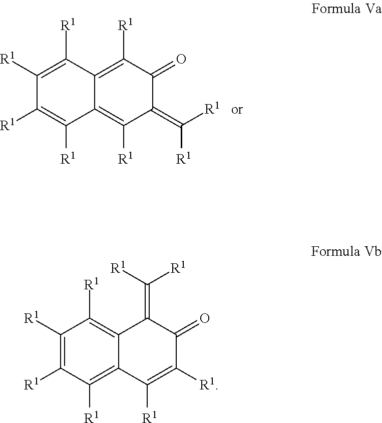

27. The method of claim 25 wherein the precursor compound is irradiated in the presence of the polarized olefin.

28. The method of claim 27 wherein irradiating the precursor compound comprises pattern-wise irradiating the substrate to provide a pattern-wise labeled surface of the substrate.

29. The method of claim 25 wherein conditions effective to form the o-quinone methide comprise irradiation of an aqueous solution, suspension, or dispersion of the precursor compound.

30. A precursor compound having the formula: 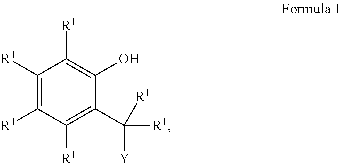
wherein:
each R¹ is independently H, halogen, or an organic group;
Y is OR⁵, NR⁵₂, NR⁵₃⁺ (Z_{1/q})⁻ wherein Z is an anion having a negative charge of q;
each R⁵ is independently H or an organic group;
optionally, two or more R¹ groups may be combined to form one or more rings;
optionally, two or more R⁵ groups may be combined to form one or more rings; and
with the proviso that at least one R¹ comprises a detectable label or a linker group attached to a surface of a substrate.

31. The precursor compound of claim 30 having one of the formulas:

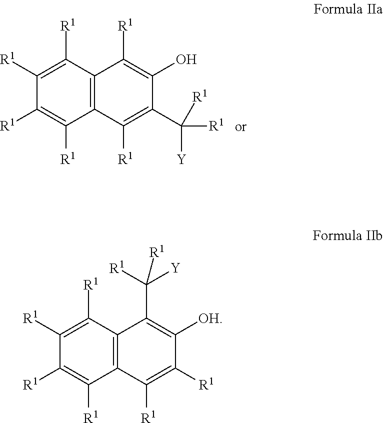

32. An o-quinone methide having the formula: 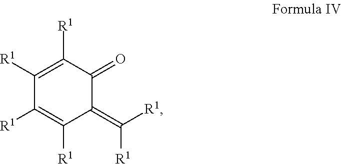
wherein each R¹ is independently H, halogen, or an organic group, with the proviso that at least one R¹ comprises a detectable label or a linker group attached to a surface of a substrate; and wherein optionally two or more R¹ groups may be combined to form one or more rings.

33. The o-quinone methide of claim 32 wherein the o-quinone methide is an o-naphthoquinone methide of one of the formulas: [chemical structure]
